SEQUENCE LISTING (8 sequences)

SEQ ID NO: 1 - DNA, 21 bp, Artificial Sequence
  non-variant detection probe
ccttgtccttcacgccagcga

SEQ ID NO: 2 - DNA, 25 bp, Artificial Sequence
  variant detection probe
accttgtccttcatgccagcgaaat

SEQ ID NO: 3 - DNA, 20 bp, Artificial Sequence
  forward primer
tcgagatcaaccccgactgt

SEQ ID NO: 4 - DNA, 17 bp, Artificial Sequence
  reverse primer
aacgggtcaggcatgca

SEQ ID NO: 5 - DNA, 20 bp, Artificial Sequence
  primer
ctcatcaccatcgagatcaa

SEQ ID NO: 6 - DNA, 20 bp, Artificial Sequence
  primer
ccaggtctgacaacgggtca

SEQ ID NO: 7 - DNA, 3651 bp, Homo sapiens
agtattgctgttcagatagcctttatttgggtatatattctacactgtttttaaatatggagagtaaccaaaatggcccattatctgaccacacaaatactagtagtcattatagataaaccatagcagataaataatagtaaacaaagcaacaggctgtgtcattggaaatccccaccatgaagaaaggagcaaggtgaaaacttctggctgcttcaggtcatgcatggtccctctccaccatcgttccccctgtcatcttcctgccagaataaggaccctggtaccttagggaagcaccatctcttgttttttccccacgagccctgtgggtcatggcacgtcctgccccgctgggaaaacacagtgggccacgggtttccctgcaggcctggacccttcccaagggtagcagcagaaggcagcacgattcccactcctgcagctgtgacagggcacccccactgtcactgagccctgcaccgggttccatcacctgctcggggctctgcctttggccttttcctgtgaactgcatgttggccactgtacctatctgtctctcatctttttttcttacgggtttgggtatgttcttggtaaaccagcccttggtcttacacatcatttccaaggtactaaggactcttcaggggaaatacaacttgagcagagtggttccctcctcttgtggttcacaaggtgcaggtgcacacacacataccacagggcagtgtgacaggaccagagactgcccctggggtccctggctgggggacactagtagggatgtcccttgcctctctgaggccttctgctgtctcttctgaggccggaaaggcgaagcactgccctcgccctgctagggaaggctcaggccaggctggccctatccggggaaggggctcaggtatctggaccttggtcatcgccaggttagggtttatgttgatgattatccaaaggcaaaattgatttccacagaaataacatctgctttgctgccgagccagaggagaccccagacccctcccgcagccagagggctggagcctgctcagaggtgctttgaaggtgagttggccaacggaagccggggcagtgccagggtgggacagaagaggcacacacctgctctgtctacccgagggcaccagagggcacgagaaggctggctccctggcgctgacacgtcaggcaactgaggcacaaggctggcatttctgaaccttgcccctctgcgaacacaagggggcgatggtggcactccaagcaaaggggcgtgtgggtgctgcaggaggagcacagagcactggcgcccctcccctcccgccctgcagatgccggaggccccgcctctgctgttggcagctgtgttgctgggcctggtgctgctggtggtgctgctgctgcttctgaggcactggggctggggcctgtgccttatcggctggaacgagttcatcctgcagcccatccacaacctgctcatgggtgacaccaaggagcagcgcatcctgaaccacgtgctgcagcatgcggagcccgggaacgcacagagcgtgctggaggccattgacacctactgcgagcagaaggagtgggccatgaacgtgggcgacaagaaaggttggggttccgggccagcaggtgctcagctctgggacagggacccaggaccaggcatcaaatcccgtgcctggggatccaagttcccctctctccacctgtgctcacctctcctccgtccccaaccctgcacaggcaagatcgtggacgccgtgattcaggagcaccagccctccgtgctgctggagctgggggcctactgtggctactcagctgtgcgcatggcccgcctgctgtcaccaggggcgaggctcatcaccatcgagatcaaccccgactgtgccgccatcacccagcggatggtggatttcgctggcgtgaaggacaaggtgtgcatgcctgacccgttgtcagacctggaaaaagggccggctgtgggcagggcgggcatgcgcactttgatcctccccaccaggtgttcacaccacgttcactgaaaacccactatcaccagggtcatcccagaaccctaaagaaaactgatgaatgcttgtatgggtgtgtaaagatggcctcctgtctgtgtgggcgtgggcactgacaggcgctgttgtataggtgtgtagggatggcctcctgtctgtgaggacgtgggcactgacaggcgctgttccaggtcacccttgtggttggagcgtcccaggacatcatcccccagctgaagaagaagtatgatgtggacacactggacatggtcttcctcgaccactggaaggaccggtacctgccggacacgcttctcttggaggtgagccccaaccaggatggcatccgtgccagctgctgcccagagcccattcagtcagcctcagcctctccaaagagccaggcattccagtagagccctgtgtggacacagctcgctctggaggcaccacctgaggtctgggagtgtgggggactgaggaggccctgtggtgggtggagatgggtggggagctgggccaggggctggctgggtggcctgttgggaactggggagccaagcggtccctgtcctcacggggcccatgttctgaaggtggcacccaagtcttgtacagtcctttcctgcaggagtcacgctgggcaggaagtggaaacctggccccaggggctaggcacaggcagtggtgccgtggcctagtgaggagcacccatcctggtttggggcaggttctctgggcacctctgacctctcacctcccccaccccccggtctgtttgcaggaatgtggcctgctgcggaaggggacagtgctactggctgacaacgtgatctgcccaggtgcgccagacttcctagcacacgtgcgcgggagcagctgctttgagtgcacacactaccaatcgttcctggaatacagggaggtggtggacggcctggagaaggccatctacaagggcccaggcagcgaagcagggccctgactgcccccccggcccccctctcgggctctctcacccagcctggtactgaaggtgccagacgtgctcctgctgaccttctgcggctccgggctgtgtcctaaatgcaaagcacacctcggccgaggcctgcgccctgacatgctaacctctctgaactgcaacactggattgttcttttttaagactcaatcatgacttctttactaacactggctagctatattatcttatatactaatatcatgttttaaaaatataaaatagaaattaagaatctaaatatttagatataactcgacttagtacatccttctcaactgccattcccctgctgcccttgacttgggcaccaaacattcaaagctccccttgacggacgctaacgctaagggcggggccctagctggctgggttctgggtggcacgcctggcccactggcctcccagccacagtggtgcagaggtcagccctcctgcagctaggccaggggcacctgttagccccatggggacgactgccggcctgggaaacgaagaggagtcagccaagcattcacacctttctgaccaagcaggcgctggggacaggtggacccgcagcagcaccagccc

SEQ ID NO: 8 - protein, 271 aa, Homo sapiens
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

(19) United States
(12) Patent Application Publication
(10) Pub. No.: US 2006/0166264 A1
(43) Pub. Date: Jul. 27, 2006

(54) Effect of COMT genotype on frontal lobe function

(76) Inventors: Daniel R. Weinberger, Washington, DC (US); Michael F. Egan, Chevy Chase, MD (US); Terry E. Goldberg, Bethesda, MD (US); Joseph H. Callicott, Bethesda, MD (US); David Goldman, Potomac, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/395,043

(22) Filed: Mar. 31, 2006

Related U.S. Application Data
(63) Continuation of application No. 10/144,000, filed on May 10, 2002.
(60) Provisional application No. 60/290,565, filed on May 11, 2001.

(51) Int. Cl. C12Q 1/68

(52) U.S. Cl. 435/6

(57) ABSTRACT

The invention provides a method of detecting impaired prefrontal cognitive function in an individual by determining the individual's COMT genotype, and associating a high activity Val allele with impaired prefrontal cognitive function (and a low activity Met allele with enhanced prefrontal cognitive function).

### RELATED APPLICATIONS

This application is a continuation of application No. Ser. 10/144,000, filed May 10, 2002, which claims benefit of U.S. Provisional Application No. 60/290,565, filed May 11, 2001, both of which are hereby expressly incorporated by reference in their entireties.

### FIELD OF THE INVENTION

The invention provides a method of detecting impaired prefrontal cognitive function in an individual by determining the individual's COMT genotype, and associating a high activity Val allele with impaired prefrontal cognitive function (and a low activity Met allele with enhanced prefrontal cognitive function).

### BACKGROUND OF THE INVENTION

Schizophrenia is a complex genetic disorder characterized by chronic psychosis, cognitive impairment, and functional disability. Linkage studies have implicated several possible susceptibility loci, including regions on chromosomes 1q, 6p, 8p, 13q and 22q (Brzustowicz, L. M. et al. 2000 Science 288:678-82; Straub, R. E. et al. 1995 Nat Genet 11:287-93; Pulver, A. E. et al. 1994 Am J Med Genet 54:36-43). Attempts to replicate these findings have met with limited success, perhaps due to the weak effects of susceptibility loci and limited power of linkage (Risch, N. & Merikangas, K. 1996 Science 273:1516-7; Risch, N. 1990 Am J Hum Genet 46:222-8). Of genes mapped to 22q11, a common functional polymorphism of catechol-O-methyltransferase (COMT), a methylation enzyme that metabolizes released dopamine (Weinshilboum, R. M. et al. 1999 Annu Rev Pharmacol Toxicol 39:19-52), has been a popular candidate because of the long hypothesized role of dopamine in schizophrenia (Carlsson, A., et al. 2000 Brain Res Brain Res Rev 31:342-349). Although two family-based association studies using the transmission disequilibrium test (TDT) have provided evidence for a role of COMT in schizophrenia (Kunugi, H. et al. 1997 Psychiatr Genet 7:97-101; Li, T. et al. 1996 Psychiatr Genet 6:131-3; Li, T. et al. 2000 Mol Psychiatry 5:77-84), several small case-control association studies of COMT alleles have been negative, and it has been unclear how either protein variation would increase risk for schizophrenia (Karayiorgou, M. et al. 1998 Biol Psychiatry 43:425-31; Palmatier, M. A. et al. 1999 Biol Psychiatry 46:557-67).

### SUMMARY OF THE INVENTION

Abnormalities of prefrontal cortical function are prominent features of schizophrenia and have been associated with genetic risk, suggesting that susceptibility genes for schizophrenia may impact on the molecular mechanisms of prefrontal function. A potential susceptibility mechanism involves regulation of prefrontal dopamine, which modulates the response of prefrontal neurons during working memory. We examined the relationship of a common functional polymorphism [Val^{108/158}Met] in the catechol-O-methyltransferase (COMT) gene, (which accounts for a four-fold variation in enzyme activity and dopamine catabolism), with both prefrontally mediated cognition and prefrontal cortical physiology. In 175 patients with schizophrenia, 219 unaffected siblings, and 55 controls, COMT genotype was related in allele dosage fashion to performance on the Wisconsin Card Sorting test of executive cognition and explained 4% of variance (p=0.001) in frequency of perseverative errors. Consistent with other evidence that dopamine enhances prefrontal neuronal function, the load of the low activity Met allele predicted enhanced cognitive performance. We then examined the effect of COMT genotype on prefrontal physiology during a working memory task in three separate subgroups (n=11 to 16) assayed with fMRI. Met allele load consistently predicted a more efficient physiological response in prefrontal cortex. Finally, in a family based association analysis of 104 trios, a significant increase in transmission of the Val allele to the schizophrenic offspring was observed. These data indicate that the COMT Val allele, because it increases prefrontal dopamine catabolism, impairs prefrontal cognition and physiology, and by this mechanism increases risk for schizophrenia.

In one embodiment, the invention provides a method of detecting impaired prefrontal cognitive function in an individual by determining the individual's COMT genotype, and associating a high activity Val allele with impaired prefrontal cognitive function (and a low activity Met allele with enhanced prefrontal cognitive function).

In another embodiment, the invention provides a method of detecting impaired prefrontal cognitive function in an individual as indicative of a susceptibility to, or the presence of, a human condition that involves deficits in prefrontal cognitive function by determining the individual's COMT genotype, and associating a high activity Val allele with impaired prefrontal cognitive function as indicative of a susceptibility to, or the presence of, the human condition (and a low activity Met allele with enhanced prefrontal cognitive function as not indicative of a susceptibility to, or the presence of, the human condition).

In yet another embodiment, the invention provides a method of detecting impaired prefrontal cognitive function in an individual as predictive of improved prefrontal cognitive function upon administration of a COMT inhibitor or its pharmaceutically acceptable salt or ester by determining the individual's COMT genotype, and associating a high activity Val allele with impaired prefrontal cognitive function as predictive of improved prefrontal cognitive function upon administration of a COMT inhibitor or its pharmaceutically acceptable salt or ester (and a low activity Met allele with enhanced prefrontal cognitive function as not predictive of improved prefrontal cognitive function upon administration of a COMT inhibitor or its pharmaceutically acceptable salt or ester).

In some embodiments, the human condition is a member selected from the group consisting of Parkinson's Disease, AIDS, normal aging, brain injury, alcoholism, schizophrenia, depression, obsessive-compulsive disorder, attention deficit hyperactivity disorder, autism, impulse control disorder, addiction, Alzheimer's disease and other forms of dementia, mental retardation, and normal cognition.

Because COMT is a susceptibility gene for schizophrenia, and because schizophrenia appears to involve at least several interacting genes, we anticipate that geneticists will use COMT genotype to find other susceptibility genes based on our finding. We predict that subjects with the Val allele are likely to have a markedly greater risk for schizophrenia if they have alleles of other genes that impair prefrontal function and physiology. Such epistatic genetic interactions are very difficult to find de novo, but this discovery process is facilitated and enhanced using COMT as a starting point.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows WCST perseverative error t scores (±standard error) by genotype for each group (population mean=50, SD=10, lower scores indicate worse performance.) Main effect of genotype F=4.93, df=2,224, p=0.008.

FIG. 2 shows effect of COMT genotype on fMRI activation during the 2-back working memory task. Regions showing a significant effect of genotype on fMRI activation (voxelwise p<0.005) are in red (shown clockwise from the upper left in right lateral, left lateral, right medial, and left medial views, respectively.) In dorsolateral prefrontal cortex (PFC) (e.g. Brodmann area (BA) 46; x=58, y=32, z=12; cluster size=47; Z=2.55) and anterior cingulate (e.g. BA 32; x=6, y=60, z=8; cluster size=77; Z=2.36), Val/Val individuals showed greater fMRI response (and by inference, greater inefficiency, as performance is similar) than Val/Met individuals who have greater activation than Met/Met individuals. Post hoc analysis of genotype group contrasts confirmed these significant relationships in dorsolateral prefrontal and cingulate cortices across all groups.

FIG. 3 shows effect of COMT genotype on fMRI activation during the 2-back working memory task in a second group of subjects. Again, Val/Val individuals showed greater activation (and by inference, greater inefficiency) than Val/Met individuals who were less efficient than Met/Met individuals in the dorsal PFC and several other locales.

### DETAILED DESCRIPTION OF THE INVENTION

One approach that may improve power to find genes for complex disorders, such as schizophrenia, is to target biological traits found in ill subjects and their unaffected relatives, so-called intermediate phenotypes, rather than clinical diagnosis (Freedman, R. et al. 1997 PNAS USA 94:587-92; Kremen, W. S. et al. 1994 Schizophr Bull 20:103-19). Such traits may be more directly related to the biological effects of susceptibility genes. Abnormal function of the prefrontal cortex, a cardinal aspect of schizophrenia, may also represent an intermediate phenotype related to genetic risk for schizophrenia (Cannon, T. D. et al. 2000 Am J Hum Genet 67:369-382; Goldberg, T. E. et al. 1990 Arch Gen Psychiatry 47:1066-72). Stable deficits in cognitive fimctions referable to the dorsolateral prefrontal cortex and cortical physiological abnormalities during performance of such tasks have been consistently reported in studies of schizophrenia (Weinberger, D. R. et al. 1986 Arch Gen Psychiatry 43:114-24; Carter, C. S. et al. 1998 Am J Psychiatry 155:1285-7; Manoach, D. S. et al. 1999 Biol Psychiatry 45:1128-37; Callicott, J. H. et al. 2000 Cereb Cortex 10:1078-1092; Goldberg, T. E. & Weinberger, D. R. 1988 Schizophr Bull 14179-83; Park, S. et al. 1995 Arch Gen Psychiatry 52:821-8). Recent evidence indicates that healthy siblings of patients, including monozygotic (MZ) co-twins, show similar cognitive and physiological abnormalities (Kremen, W. S. et al. 1994 Schizophr Bull 20:103-19; Cannon, T. D. et al. 2000 Am J Hum Genet 67:369-382; Goldberg, T. E. et al. 1990 Arch Gen Psychiatry 47:1066-72; Park, S. et al. 1995 Arch Gen Psychiatry 52:821-8; Callicott, J. et al. 1998 Neuroimage 7:S895; Egan, M. et al. 2001 Biol Psychiatry 50:98-107).

Prefrontal deficits are also appealing phenotypes for genetic studies because the molecular mechanisms underlying such deficits have been sufficiently clarified to permit a hypothesis-driven test of candidate fumctional polymorphisms (Lidow, M. S. et al. 1998 Trend Pharm Sci 19:136-140; Gao, W. J. et al. 2001 PNAS USA 98:295-300). Electrophysiological studies in primates (Sawaguchi, T. & Goldman-Rakic, P. S. 1991 Science 251:947-50; Williams, G. V. & Goldman-Rakic, P. S. 1995 Nature 376:572-5) and rodents (Seamans, J. K. et al. 1998 J Neurosci 18:1613-21), and neuroimaging studies in humans (Daniel, D. G. et al. 1991 J Neurosci 11:1907-17; Mattay, V. S. et al. 1996 J Neurosci 16:4816-22), have shown that dopamine plays an important role in modulating the activity of prefrontal circuitry during performance of working memory tasks. While there are many proteins involved in the biological actions of dopamine, catechol-O-methyltransferase (COMT), because it metabolizes released dopamine, may be an important factor during such prefrontally mediated tasks. Despite its widespread distribution in nondopaminergic neurons and glia, pharmacological studies have shown that catabolic flux of synaptic dopamine through the COMT pathway is characteristic of the prefrontal cortex in contrast to the striatum (Karoum, F. et al. 1994 J Neurochem 63:972-9). Studies of COMT knockout mice, similarly, have demonstrated that dopamine levels are increased only in prefrontal cortex (Gogos, J. A. et al. 1998 PNAS USA 95:9991-6) and that memory performance is enhanced (Kneavel, M. et al. 2000 Society for Neuroscience 30th Annual Meeting, New Orleans, 571.20 abstr.). This regionally selective effect of COMT may be because, in contrast to striatum, in prefrontal cortex dopamine transporters are expressed in low abundance and not within synapses (Lewis, D. A. et al. 1998 Adv Pharmacol 42:703-6; Sesack, S. R. et al. 1998 J Neurosci 18:2697-708). As a consequence, released synaptic dopamine appears to be inactivated by difflusion, receptor internalization, and COMT degradation. These findings support the notion that variation in COMT activity may have neurobiological effects specific to the prefrontal cortex.

The COMT gene contains an evolutionarily recent G to A missense mutation that translates into a substitution of methionine (Met) for valine (Val) at codon 108/158 [Val^{108/158}Met] (Genfank accession no. Z26491). The enzyme containing methionine is unstable at 37° C. and has ¼ of the activity of the enzyme containing valine (Lotta, T. et al. 1995 Biochemistry 34:4202-10). The alleles are codominant, as heterozygous individuals have enzyme activity that is midway between homozygote individuals (Weinshilboum, R. M. et al. 1999 Annu Rev Pharmacol Toxicol 39:19-52). Thus, genetically determined variations in COMT activity affect prefrontal cortical activity, especially during executive and working memory tasks. We hypothesized that the high activity Val allele, because it leads to increased dopamine catabolism, would be associated with relatively compromised prefrontal ftunction, and, by virtue of this effect, increase risk for schizophrenia.

To test these hypotheses, we studied prefrontal executive cognition and physiology in control subjects, patients with schizophrenia, and their unaffected siblings. To measure executive cognition and working memory, we used the Wisconsin Card Sorting Test (WCST). Deficits in WCST performance are enduring and core features of schizophrenia and predict long-term disability, independent of other cognitive deficits (Weinberger, D. R. et al. 1986 Arch Gen Psychiatry 43:114-24; Goldberg, T. E. & Weinberger, D. R. 1988 Schizophr Bull 14:179-83); healthy siblings of patients with schizophrenia also perform abnormally on it (Egan, M. et al. 2001 Biol Psychiatry 50:98-107; Faraone, S. V. et al. 1995 J Abnorm Psychol 104:286-304). Functional neuroimaging studies have found that the WCST activates the dorsolateral prefrontal cortex (Weinberger, D. R. et al. 1986 Arch Gen Psychiatry 43:114-24; Berman, K. F. et al. 1995 Neuropsychologia 33:1027-46) and that dopamimetic drugs improve performance on this task in patients with schizophrenia and enhance the signal to noise of the prefrontal physiological response (Daniel, D. G. et al. 1991 J Neurosci 11:1907-17; Mattay, V. S. et al. 1996 J Neurosci 16:4816-22).

To assay prefrontal physiology, we used functional magnetic resonance imaging (FMRI) while subjects performed the N-back task. This task has been shown to activate dorsolateral prefrontal cortex, as well as a distributed cortical working memory network (Callicott, J. H. et al. 2000 Cereb Cortex 10:1078-1092; Cohen, J. D. et al. 1997 Nature 386:604-8). In studies of patients with schizophrenia who perform relatively well on the N-back and similar tasks, fMRI activation of dorsolateral prefrontal cortex is “inefficient”, i.e. there is excessive activity for a given level of performance (Manoach, D. S. et al. 1999 Biol Psychiatry 45:1128-37; Callicott, J. H. et al. 2000 Cereb Cortex 10:1078-1092). Similar fMRI results have been described in their unaffected siblings (Callicott, J. et al. 1998 Neuroimage 7:S895), suggesting that inefficient prefrontal information processing is related to genetic risk for schizophrenia. Using the N-back fMRI paradigm, Mattay et al. recently reported analogous inefficiency in hypodopaminergic patients with Parkinson's disease (Mattay, V. S. et al. 2000 Society for Neuroscience, 30th Annual Meeting Book of Abstracts, Vol. 26, Pt 1, 746). In contrast, the efficiency of the N-back fMRI response in dorsolateral prefrontal cortex is enhanced by the dopamimetic drug, amphetamine, in healthy individuals whose performance remains stable (Mattay, V. S. et al. 2000 Neuroimage 12:268-75).

Thus, deviations of prefrontal physiology can be appreciated with this in vivo fMRI assay even if there is compensation at the level of performance accuracy, and changes in cortical dopaminergic function impact on physiological efficiency during this task. We hypothesized, therefore, that COMT genotype would affect the efficiency of the prefrontal fMRI response during this task and predicted an allele dosage relationship with activation, with Val/Val individuals being least efficient.

Effect of COMT and Increased Risk for Schizophrenia

Demographic data are presented in Table 1. Briefly, siblings and controls were well matched on age, gender, education, IQ and the Wide Range Achievement Test (WRAT). There was no difference between patients receiving typical and atypical neuroleptic treatment on any cognitive variable. History of alcohol abuse and dependence did not affect any cognitive measure in this study, most likely because subjects with recent or prolonged abuse or dependence were excluded (Egan, M. et al. 2000 Am J Psychiatry 157:1309-1316).

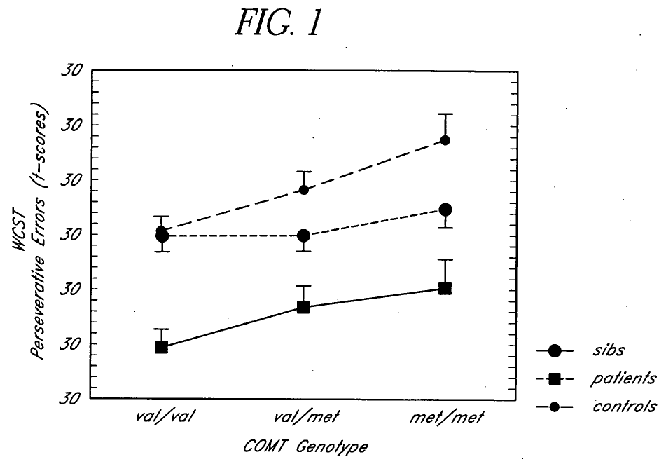

Patients and siblings scored significantly worse on the WCST compared to the control group (Table 1), as previously reported (Egan, M. et al. 2001 Biol Psychiatry 50:98-107; Faraone, S. V. et al. 1995 J Abnorm Psychol 104:286-304) (F=29.6, df=2,440, p<0.00001). An ANOVA for all groups revealed a significant effect of COMT genotype on WCST performance (F=6.00, df=2,440, p=0.003) with no group by genotype interaction (F=1.40, df=4,440, p=0.23, FIG. 1). A second ANOVA including only patients and controls also detected a significant effect of genotype (F=4.93, df=2,224, p=0.008). Post hoc analysis showed that subjects with the Val/Val genotype performed worse than those with the Val/Met and Met/Met genotypes (p<0.002). In contrast, no genotype effect was seen on tasks of general academic ability, e.g., WRAT reading scores or IQ, and no differences were seen between genotype groups in other demographic measures (Table 2).

TABLE 1
Demographics¹

| | Patients | Siblings | Controls |
|---|---|---|---|
| Variable | (n = 175) | (n = 219) | (n = 55) |
| Age | 36.1 (8.5) | 35.6 (8.8) | 33.9 (9.2) |
| Gender (M/F) | 138/37^{2,3} | 97/122 | 23/32 |
| Education years | 13.7^{2,3} (2.1) | 15.5 (2.5) | 15.7 (2.5) |
| WRAT | 102.0^{2,3} (12.1) | 106.3 (11.2) | 107.3 (11.4) |
| IQ | 92.8^{2,3} (13.1) | 107.4 (10.6) | 109.1 (11.5) |
| WCST perseverative | 37.6^{2,3} (12.6) | 45.2 (9.5)² | 49.4 (9.0) |
| errors | | | |

¹Means ± SD.
²Significantly different compared to controls (p < 0.05).
³Significantly different compared to siblings (p < 0.05).

TABLE 2
Demographics by Genotype for Patients and Controls

| | Patients | | | Controls | | |
|---|---|---|---|---|---|---|
| | Val/Val | Val/Met | Met/Met | Val/Val | Val/Met | Met/Met |
| Age | 37.1 (8.3) | 35.7 (8.1) | 35.1 (8.3) | 34.5 (10.5) | 33.7 (10.0) | 34.2 (9.5) |
| Gender | 49/13 | 68/17 | 21/7 | 6/9 | 10/20 | 7/3 |
| (M/F) | | | | | | |
| Education | 13.9 (2.0) | 13.6 (2.0) | 13.5 (2.6) | 16.3 (2.5) | 15.8 (2.3) | 15.8 (2.6) |
| Years | | | | | | |
| WRAT | 102.1 (10.7) | 102.4 (11.4) | 100.9 (13.4) | 108.0 (9.1) | 106.8 (10.6) | 107.4 (6.0) |
| IQ | 89.9 (13.7) | 94.3 (12.0) | 94.5 (12.6) | 111.5 (8.7) | 107.3 (9.2) | 110.4 (8.8) |

Means ± SD. Within each group (patients or controls), there is no significant difference between genotype for any variable.

Using multiple regression, the number of Met alleles was parametrically related to perseverative errors t scores (r²=0.041, t(228)=3.29, p=0.001). COMT genotype accounted for 4.1% of the variance in performance. Because prior reports have found an effect of gender on COMT expression in animal models (Gogos, J. A. et al. 1998 PNAS USA 95:9991-6), we added gender into both the ANOVA and multiple regression analyses. There was no effect of gender or gender by genotype interaction. To exclude other possible spurious effects, we added diagnosis, age, gender, and education to a stepwise multiple regression analysis. This resulted in a small decrease in the r² for the COMT effect but its significance at entry remained high (increment in adjusted r²=0.024, p=0.003). Using the family-based quantitative sib transmission disequilibrium test (TDT), a trend was seen for a COMT genotype effect on WCST performance (F=2.36, df=2,159, p<0.10). Using 19 polymorphic genetic markers, no evidence for population stratification was found between Val/Val and Met/Met groups in patients or controls (omnibus χ²=113.5, df=112, p=0.44).

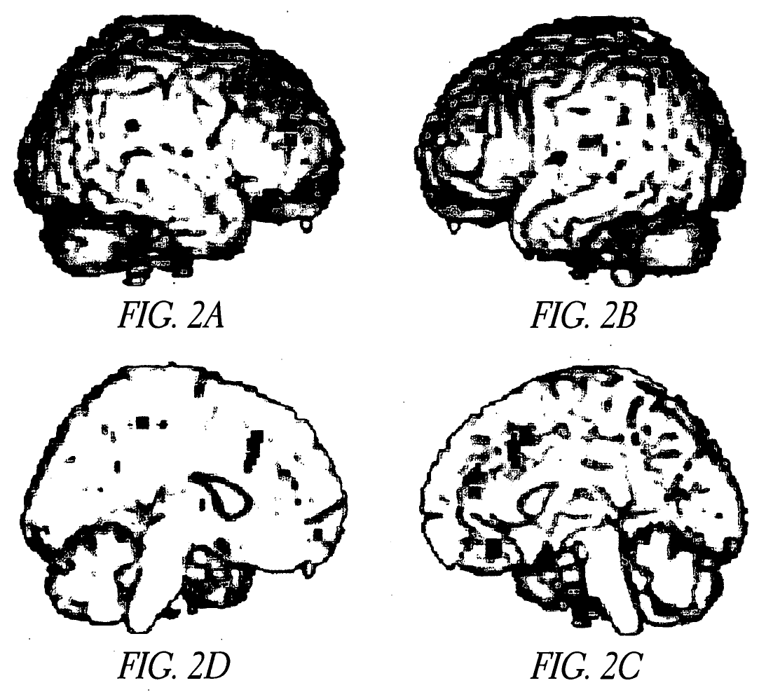

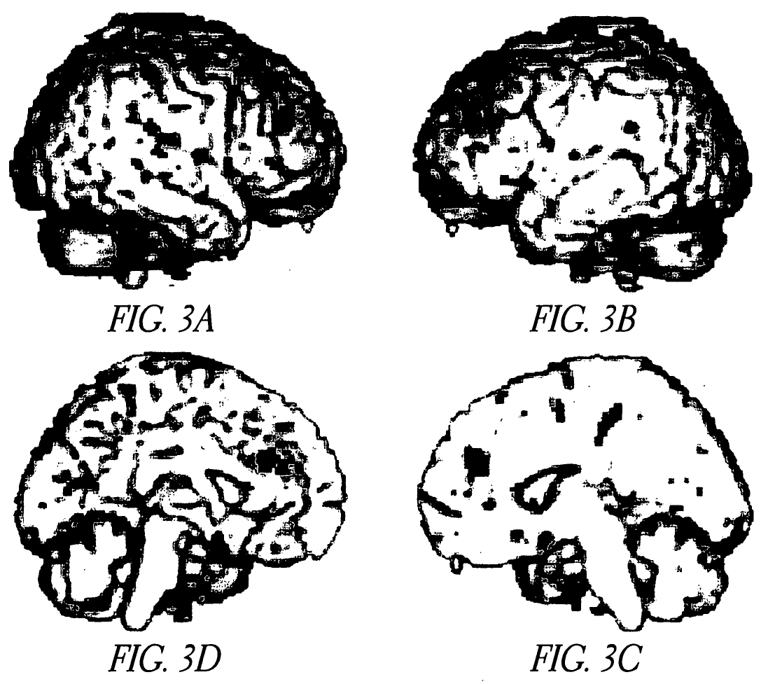

FIGS. 2 and 3 show the effect of COMT allele load on the fMRI response during the 2-back version of the N-back task in the two groups of siblings. The first group (FIG. 2) consisted of five Met/Met individuals, six Val/Met individuals, and five Val/Val individuals. The genotype subgroups used did not differ in mean age, gender, education, handedness, or performance accuracy. The second group (FIG. 3) consisted of three Met/Met, five Met/Val, and three Val/Val individuals; these genotype subgroups did not differ significantly in age, education, gender, handedness or performance accuracy. In both groups, locales in dorsolateral prefrontal and cingulate cortices show the predicted genotype effects, with Val/Val individuals having the greatest response (i.e., being least efficient), followed by Val/Met and then Met/Met individuals. Similar results were seen in the patient group as well.

We next addressed the possibility that in the 104 family trios, the COMT Val allele is a risk factor for schizophrenia, per se. A total of 126 transmissions were counted from heterozygous parents to probands. The Val allele was transmitted 75 times, compared to 51 transmissions of the Met allele. These proportions are different from that predicted by random assortment (χ²=4.57; p=0.03) and indicate that the COMT Val allele is weakly associated with schizophrenia. The odds ratio for the Val/Val genotype is 1.5. Unaffected siblings (n=117) had 77 Val transmissions and 87 Met transmissions, indicating that meiotic segregation distortion is not present. Monte Carlo simulation of 10,000 TDT replicates confirmed that our result would occur at the p<0.04 level of significance. In the case control analysis, no significant differences in allele (χ²=0.92; df=1; p=0.34) or genotype (χ²=1.25; df=2; p=0.54) frequencies were seen comparing patients and controls (Table 3), similar to most (Karayiorgou, M. et al. 1998 Biol Psychiatry 43:425-31; Daniels, J. K. et al. 1996 Am J Psychiatry 153:268-70; Chen, C. H. et al. 1997 Biol Psychiatry 41:985-7; Strous, R. D. et al. 1997 Biol Psychiatry 41:493-5), but not all (de Chaldee, M. et al. 1999 Am J Med Genet 88:452-7; Ohmori, O. et al. 1998 Neurosci Lett 243:109-12) earlier case-control studies.

TABLE 3
Distribution of Genotypes and Alleles.

| Genotype | Patients (n = 175) | Siblings (n = 219) | Controls (n = 55) |
|---|---|---|---|
| Val/Val | 62 (35%) | 69 (31%) | 15 (27%) |
| Val/Met | 85 (49%) | 114 (51%) | 30 (55%) |
| Met/Met | 28 (16%) | 39 (18%) | 10 (18%) |
| Frequency¹ Val | 0.60 ± 0.03 | 0.57 ± 0.02 | 0.54 ± 0.03 |
| Frequency Met | 0.40 ± 0.03 | 0.43 ± 0.02 | 0.46 ± 0.03 |

¹±Standard error.

We report several convergent findings that implicate an effect of COMT genotype on prefrontal cortical fluction and, as a result, on increased risk for schizophrenia. First, COMT genotype is specifically associated with level of performance on a neuropsychological test of executive cognition that is related to function of prefrontal cortex, but not with general intelligence. This effect of COMT is independent of psychiatric diagnosis and explains 4.1% of the variance on the WCST. The high activity Val allele is associated with a reduction in performance compared with the Met allele. Second, Val allele load is related to reduced “efficiency” of the physiologic response in the dorsolateral prefrontal cortex during performance of a simple working memory task in three cohorts studied with fMRI. Neural net modeling of the effects of dopamine on working memory circuits predicted that reductions in synaptic dopamine would reduce signal to noise ratios, thus reducing efficiency (Servan-Schreiber, D. et al. 1990 Science 249:892-5). This prediction was recently confirmed in an fMRI study of patients with Parkinson's disease (Mattay, V. S. et al. 2000 Society for Neuroscience, 30th Annual Meeting Book of Abstracts, Vol. 26, Pt. 1, 746). It is also consistent with the effect of the Val allele observed in our fMRI data. Mechanism of action-wise, these convergent findings suggest that the COMT Val allele, presumably by compromising the postsynaptic impact of the evoked dopamine respohse, may reduce signal to noise in prefrontal neurons and thereby alter working memory function. Third, the Val allele is transmitted slightly more often (p<0.04) to probands with schizophrenia. The association of the Val allele with schizophrenia indicates that this allele, by virtue of its physiological effect on prefrontal information processing, increases susceptibility to schizophrenia.

This proposed genetic/neurophysiological mechanism is consistent with prior studies of the neurobiology of schizophrenia. As described above, deficits in prefrontal function are core manifestations of schizophrenia and are related to genetic risk for schizophrenia (Cannon, T. D. et al. 2000 Am J Hum Genet 67:369-382; Goldberg, T. E. et al. 1990 Arch Gen Psychiatry 47:1066-72; Egan, M. et al. 2001 Biol Psychiatry 50:98-107). Neuroimaging and postmortem studies have found evidence of reduced dopaminergic innervation of dorsolateral prefrontal cortex in patients with schizophrenia (Weinberger, D R. et al. 1986 Arch Gen Psychiatry 43:114-24; Weinberger, D. R. et al. 1988 Arch Gen Psychiatry 45:609-15; Akil, M. et al. 1999 Am J Psychiatry 156:1580-9). Thus, the COMT Val allele, by imposing an additional adverse load specifically on prefrontal function, might add to or interact with other causes of prefrontal malfunction in those at risk for schizophrenia and thereby increase their susceptibility. However, the effect of COMT genotype on prefrontal function is small; indeed, it was not significant in the cohort of siblings. This latter negative finding could be due to siblings being a mixed group, in terms of other genetic risk and protective factors. Val/Val siblings who have no psychiatric disorder, for example, could have protective factors positively affecting prefrontal cortical function, otherwise they might themselves have schizophrenia.

With an odds ratio of 1.5, the effect of the Val/Val genotype acting alone on diagnosis is weak. Indeed, a 4.1% variation in prefrontal function by itself may not pose much of a risk for behavioral decompensation. This risk, however, represents an average effect across many individuals. The effect of COMT genotype within any particular individual could be large or small, depending on a variety of background factors. Thus, a gene such as COMT could have an important clinical effect in combination with other genes and environmental factors, and could be of value in identifying such factors, especially if their effects are nonadditive. Nevertheless, it seems possible or even likely that most susceptibility genes for schizophrenia will either have a relatively low genotypic relative risk or will be very uncommon in the general patient population and affect only a small portion of patients (Risch, N. 1990 Am J Hum Genet 46:222-8; Riley, B. P. & McGuffin, P. 2000 Am J Med Genet 97:23-44).

While our results offer a mechanism for how the Val allele might increase susceptibility for schizophrenia, the results of genetic studies, including this one, showing linkage or association between COMT and schizophrenia are, at best, weak. Linkage studies have generally found LOD scores of 2 or less for markers near 22q11, the chromosomal region containing the COMT gene (Pulver, A. E. et al. 1994 Am J Med Genet 54:36-43; Pulver, A. E. et al. 1994 Am J Med Genet 54:44-50; Gill, M. et al. 1996 Am J Med Genet 67:40-5) (see Riley, B. P. & McGuffin, P. 2000 Am J Med Genet 97:23-44 for review). Of previously published TDT based association studies, one found a significant relationship between schizophrenia and Val transmissions (Li, T. et al. 1996 Psychiatr Genet 6:131-3); a second also reported an excess (22 vs. 13, χ²=2.31, p=0.13) of Val transmissions (Kunugi, H. et al. 1997 Psychiatr Genet 7:97-101). In an expanded sample of 198 trios, Li et al. performed a haplotype analysis and again showed a significant association with the Val allele and schizophrenia (Li, T. et al. 2000 Mol Psychiatry 5:77-84). While TDT analyses have been uniformly positive, the results of case control association studies (including our own), which have generally used small sample sizes (relative to those needed to detect a weak genetic effect), have been negative in most (Karayiorgou, M. et al. 1998 Biol Psychiatry 43:425-31; Daniels, J. K. et al. 1996 Am J Psychiatry 153:268-70; Chen, C. H. et al. 1997 Biol Psychiatry 41:985-7; Strous, R. D. et al. 1997 Biol Psychiatry 41:493-5; de Chaldee, M. et al. 1999 Am J Med Genet 88:452-7), but not all (de Chaldee, M. et al. 1999 Am J Med Genet 88:452-7) cases. These negative results are not unexpected, given the lack of power in these studies to detect alleles of minor effect.

Population stratification artifacts are an important consideration in genetic case-control analyses and might be an occult factor in our genetic effect on prefrontal function. COMT Val/Met allele frequencies differ across some ethnic groups, although this is probably not the case for the western European populations represented in our study (Palmatier, M. A. et al. 1999 Biol Psychiatry 46:557-67). Given that the predicted COMT effect on WCST performance was seen in two unrelated samples (patients and controls) and the predicted effect on cortical physiology was found in three samples, similar stratification would have to be common to all these cohorts and both phenotypes. Furthermore, the genetically distinct subpopulations would have to differ only on prefrontal measures and not on general intelligence, since genotype groups did not differ on other cognitive tests. Nevertheless, we also used two methods to test whether admixture might account for our genetic effect on cognition, a family based analysis (Allison, D. B. et al. 1999 Am J Hum Genet 64:1754-63) and genomic controls (Pritchard, J. K. & Rosenberg, N. A. 1999 Am J Hum Genet 65:220-8). The quantitative sibling TDT used with the WCST data was not significant, though with a trend p value of <0.10, but this is a random effects model with limited degrees of freedom. Using 19 unlinked polymorphic genetic markers, we found no genetic evidence for stratification. The family based TDT, which found a weakly significant association with schizophrenia, also controls for stratification (Spielman, R. S. et al. 1993 Am J Hum Genet 52:506-16).

A second possible artifact to consider is that the COMT Val/Met polymorphism is not the causative locus but is in linkage disequilibrium with another mutation. We suggest that, given 1) the strong impact of the COMT Val/Met polymorphism on COMT enzyme activity, 2) the known effects of COMT on prefrontal dopamine metabolism, and 3) the effect of dopamine on prefrontal neuronal function and working memory, the COMT Val/Met allele is the causative genetic locus for the association with prefrontal function. Using a COMT knockout mouse model, others have shown that prefrontal dopamine levels are increased (Gogos, J. A. et al. 1998 PNAS USA 95:9991-6) and that performance on a memory task is actually improved relative to the wild type animal (Kneavel, M. et al. 2000 Society for Neuroscience 30th Annual Meeting, New Orleans, 571.20 abstr.). This improvement in memory performance supports our model that the Met allele, with its reduced activity, accounts for improved prefrontal fimction, and not another nearby gene.

Finally, is it plausible that a common allele with such weak effects could increase risk for schizophrenia? In some respects, our results with COMT and schizophrenia are similar to the calpain-10 association with diabetes (Horikawa, Y. et al. 2000 Nat Genet 26:163-75), and the association of the APO e4 allele with Alzheimer's Disease, though the APO e4 effect is much greater (Roses, A. D. 1998 Ann N Y Acad Sci 855:738-43). The calpain-10 allele is found in 75% of the general population and in 80% of diabetics, a weak association that is not easily replicated across populations, and the biologic effect of the polymorphism is unknown. It is assumed that such polygenes interact with other genes and environmental factors to incrementally increase risk. The COMT Val allele is certainly not a necessary or sufficient causative factor for schizophrenia, nor is it likely to increase risk only for schizophrenia. However, its biological effect on prefrontal function and the relevance of prefrontal function for schizophrenia susceptibility implicate a mechanism by which it could increase liability for this disorder. The data presented here provide convergent evidence that the Val allele compromises prefrontal function and thereby impacts directly on the biology of schizophrenia. Despite the apparent disadvantage of the Val allele, the Met allele may increase susceptibility to other disorders, such as estrogenic cancer (Lavigne, J. A. et al. 1997 Cancer Res 57:5493-7), suggesting that a heterozygote advantage could maintain the high Met and Val allele frequencies observed in a variety of human populations. Finally, it should be noted that the COMT polymorphism affects performance and prefrontal cortical function in both ill and healthy subjects. Thus, the recent Met mutation, which has not been reported in nonhuman primates (Palmatier, M. A. et al. 1999 Biol Psychiatry 46:557-67), enhances an important component of normal human cognition, suggesting a possible role in the evolution of human brain function.

Effect of COMT in Healthy Volunteers

In the prefrontal cortex, the enzyme catechol-O-methyltransferase (COMT) is critical in the metabolic degradation of dopamine, a neurotransmitter hypothesized to influence human cognitive function. The COMT gene contains a functional polymorphism, Val158Met, which exerts a four-fold effect on enzyme activity. In this study, we genotyped Val158Met in 73 healthy volunteers who had been administered the neurocognitive test, the Wisconsin Card Sort Test (WCST). Subjects with the low activity Met allele made significantly fewer perseverative errors on the WCST as compared to subjects with the Val allele. These data are consistent with the prediction that increases in synaptic dopamine availability enhance prefrontal cortex-mediated cognition and lead to the conclusion that a functional genetic polymorphism influences one aspect of human cognitive variation.

Several lines of evidence suggest that the neurotransmitter dopamine plays an important role in the human cognition. Computational modeling studies indicate that condition in dopamine systems accounts for abnormal cognitive control in prefrontal cortex. In laboratory animals, reduced prefrontal cortical dopamine transmission is associated with impairments in cognitive performance and, in humans, pharmacological enhancement of dopaminergic activity can produce improvements in specific cognitive domains dependent on the integrity of the prefrontal cortex.

At the synaptic level, dopaminergic function is critically affected by catechol-O-methyltransferase (COMT), a major mammalian enzyme involved in the metabolic degradation of released dopamine. COMT activity accounts for more than 60% of the metabolic degradation of dopamine in the frontal cortex. We felt it was therefore plausible that genetic factors that affect COMT function may significantly influence cognition through effects on dopaminergic function.

The COMT gene contains a functional polymorphism that codes for a methionine for valine substitution at codon 158 (GenBank accession no. Z26491). The Met allele is thermolabile and is four fold reduced in enzymatic activity compare to the Val allele. As described herein, subjects with the low activity Met allele performed better (as measured by fewer perseverative errors) on a neurocognitive test, the Wisconsin Card Sort Test (WCST), than subjects with the Val allele. This relationship of genotype to cognitive performance was observed in three groups: healthy volunteers, schizophrenia patients, and the siblings of schizophrenia patients. We therefore examined a cohort of healthy volunteers who were assessed with the WCST and genotyped at the COMT Val158Met locus to test the hypothesis that subjects with the COMT Met allele would perform better on the WCST than subjects with the COMT Val allele.

Seventy-three healthy volunteers (mean age=31.3±10.2 years, 42M, 31F, 49 Caucasians, 14 Blacks, 5 Hispanics, 3 Asians, 2 mixed ethnicity) provided written informed consent and participated in the study. All subjects were free of psychiatric disorders as determined by a structured diagnostic interview and in good physical health as determined by physical exam, electrocardiogram, and laboratory testing including liver and thyroid function tests and urinalysis. All had been free of drug and alcohol abuse for at least six months. Subjects were each administered the WCST, a widely used measure of executive cognitive function that focuses on the subject's ability to generate hypotheses, establish response sets, and switch sets by sorting stimulus cards on the basis of perceptual attributes (color, form, number). The only feedback provided by the administrator is whether each response is correct or incorrect. The sorting rule is changed after 10 consecutive correct responses. Testing is completed when the subject has completed six correct categories or reached 128 trials. The main outcome measure on the WCST is the number of perseverative errors, a measure sensitive to an individual's ability to fluently shift cognitive sets.

COMT Val158Met genotypes were determined by restriction fragment length polymorphism, as described herein.

Welsh's analysis variance (ANOVA) was carried out with COMT genotype as the independent factor and number of perseverative errors (PE) as the dependent measure. This procedure was used because a test of the assumption of homogeneity of variance indicated that the variances within each group were dissimilar (F=3.42, df=2,70, p=0.04), and Welsh's ANOVA is robust to such violations. A mixed model analysis that fit separate variances for each group was then used to do paired comparisons.

The distribution of COMT genotypes was Met/Met=13, Met/Val=31, and Val/Val=29, consistent with Hardy-Weinberg expectations (χ²=0.84, df=2, p=0.67). The numbers of perseverative errors by genotype were: Met/Met=7.46±4.01, Met/Val=13.03±11.18, and Val/Val 12.21±9.08. ANOVA revealed a significant relationship between COMT Val158Met genotype and the number of perseverative errors (F=4.43, df=2, p=0.02). The Met/Met group committed significantly fewer perseverative errors than either the Met/Val group (t=2.43, p=0.02) or the Val/Val group (t=2.35, p=0.02). The difference between the performance of the Met/Val and Val/Val groups was not significant (t=0.31, p=0.75). No significant differences were observed between the three genotypic groups in age (F=0.03, df=2, p=0.97), sex (χ²=4.67, df=2, p=0.10), or ethnicity (Fisher's exact test (F.E.T.)=8.05, p=0.18).

These data provide evidence that the COMT Met allele that results in reduced dopamine metabolism is also associated with better performance on a neurocognitive task, the WCST, and lend support to the conclusion that the COMT Val158Met polymorphism influences executive cognitive performance in healthy human subjects. These data replicate and extend the results presented in the above-described study reporting a similar relationship between COMT genotype and WCST performance. This work provides additional evidence for a contribution of dopamine to human cognition and provides independent support for a role of COMT genotype in one aspect of human cognition.

Effect of COMT in Subjects with Traumatic Brain Injury

112 individuals who sustained traumatic brain injury and were evaluated at Walter Reed Medical Center gave informed consent to have their stored blood sample and neuropsychological testing results included in genetic analysis.

A 5′ nuclease (Taqman®) assay using fluoroGenic detection probes was performed based on the G1947A single nucleotide polymorphism (SNP) within exon 4 of the COMT gene (GenBank accession number Z26491). The detection oligonucleotide sequences were: 5′-FAM6-CCTTGTCCTTCAcGCCAGCGA-TAMRA-3′ (non-variant detection probe) (SEQ ID NO: 1) and 5′-Vic-ACCTTGTCCTTCAtGCCAGCGAAAT-TAMRA-3′ (variant detection probe) (SEQ ID NO: 2). The SNP is shown as a lower case letter for the non-variant G and variant A, respectively. The oligonucleotide primers used for amplification consisted of 5′-TCGAGATCAACCCCGACTGT-3′ (forward) (SEQ ID NO: 3) and 5′-AACGGGTCAGGCATGCA-3′ (reverse) (SEQ ID NO: 4). Target DNA amplification, fluorescence measurements, and allele discrimination were accomplished using a PE 7700 Sequence Detector (Perkin Elmer, Foster City, Calif.), gathering data for up to 96 samples at a time.

COMT158Val was designated as COMT*1 and COMT158Met was designated as COMT*2. Of the total sample, 37 were homozygous for COMT*1, 26 were homozygous for COMT*2, and 49 individuals were heterozygotes. ANOVA of perseverative errors on the Wisconsin Card Sorting Test (WCST) and the 3 COMT genotypes showed a significant difference between the groups (p=0.04). Post-hoc analysis ascertained a significant difference between the homozygotes, with no significant difference found between either COMT*1 homozygotes or COMT*2 homozygotes and the heterozygous individuals. Individuals homozygous for the COMT*2 allele made fewer perseverative errors on the WCST (mean=10.46), whereas those homozygous for COMT*1 made the most errors (mean=20.30). Heterozygotes made an intermediate number of errors (mean=14.35). No significance between group differences were found with other tests thought to involve executive function including animal naming, controlled oral word association and time to complete Trails B. Statistical analysis of between group differences included analysis of variance (ANOVA) and post-hoc analyses were completed using a Bonferroni correction.

N-Back Task

The genetic complexity of schizophrenia has encouraged an approach to phenotyping that attempts to deconstruct schizophrenia into heritable neurobiological/information processing elements, each of which may have a relatively simple genetic architecture. We have discovered that cognitive dysfumction in the working memory domain is one such intermediate phenotype in schizophrenia. The present study assessed the relative risk (RR) of working memory impairments and speed of information processing impairments in a large and unselected sample of schizophrenic patients, their well siblings who did not have schizophrenia spectrum diagnoses, and healthy controls (total n=250). We used the N-back task as a working memory assay as it demands information maintenance and updating over increasing loads and delays, with the explicit objective of determining heritable, neurobiologically sensitive component processes. A parametric analysis in which family membership was treated as a random factor was used to test group differences in N-back performance. Subjects were also genotyped for COMT at the Val/Met 158 locus. Based upon work described herein, we predicted that this functional polymorphism of the COMT gene would have significant effects on working memory function. Relative Risks (RRs) for impaired performance in siblings on the N-back One and Two tests were elevated 2.8 fold and 3.9-fold respectively (p<0.05). In the parametric analyses, the sibling group performed significantly worse than the normal group on the One and Two back tests, but not on measures of reaction time (RT). A significant COMT Val allele load effect was also found: the Val/Val individuals had the lowest N-back performance and Met/Met individuals had the highest performance in normal controls, siblings, and patients. The effect was also relatively specific: COMT genotype had no impact on IQ, reading ability, and visual spatial processing, nor on RT. By parsing the subcomponents of the N-back that were preferentially related to RR and the impact of COMT genotype, we were able to demonstrate that information manipulation, in the sense of rapid target selection and de-selection, but not load/delay or speed of processing, was critical to the results.

Detection of COMT Gene and Gene Products

There is one single gene for COMT, which codes for both soluble COMT (S-COMT) and membrane-bound COMT (MB-COMT) using two separate promoters. Human S-COMT contains 221 amino acids, and the molecular mass is 24.4 kDa. Human MB-COMT contains 50 additional amino acids, of which 20 are hydrophobic membrane anchors. The remainder of the MB-COMT molecule is suspended on the cytoplasmic side of the intracellular membranes. The corresponding molecular mass is 30.0 kDa.

COMT O-methylates catecholamines and other compounds with a catechol structure. As discussed above, the level of COMT enzyme activity is genetically polymorphic in human tissues with a trimodal distribution of high (COMTHH), intermediate (COMT^{HL}), and low (COMT^{LL}) activities. This polymorphism, which is caused by autosomal codominant alleles, leads to 3- to 4-fold differences in COMT activity. It has been recently shown that the molecular basis for this variation in activity is due to a transition of guanine to adenine at codon 158 of the COMT gene that results in a substitution of Val158 by Met158 in MB-COMT (or the corresponding amino acids 108 in S-COMT). The two alleles (high activity Val allele and low activity Met allele) and the three genotypes (Val/Val, Val/Met, and Met/Met) can be identified with a PCR-based restriction fragment length polymorphism analysis using the restriction enzyme NlalIl. The nucleic acid sequence for the human COMT gene is set forth in GenBank Accession Number Z26491.

Probes and primers based on the subject COMT nucleotide sequences can be used to detect transcripts or genomic sequences encoding COMT. The invention thus provides probes and primers comprising an oligonucleotide, which oligonucleotide comprises a region of nucleotide sequence that hybridizes under stringent conditions to at least approximately 12, preferably 25, more preferably 40, 50, or 75 consecutive nucleotides of sense or antisense sequence from the nucleic acid sequence for the human COMT gene set forth in GenBank Accession Number Z26491. Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, eds. Ausubel et al. 1994 N.Y. John Wiley & Sons; and Molecular Cloning A Laboratory Manual, 2nd Ed., ed. by Sambrook, Fritsch and Maniatis 1989 Cold Spring Harbor Laboratory Press). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature and salt concentration may be held constant while the other variable is changed. In a preferred embodiment, a COMT nucleic acid of the present invention will bind to a nucleic acid sequence for the human COMT gene set forth in GenBank Accession Number Z26491 or a complement thereof under moderately stringent conditions, for example, at about 2.0×SSC and about 40° C. In a particularly preferred embodiment, a COMT nucleic acid of the present invention will bind to a nucleic acid sequence for the human COMT gene set forth in GenBank Accession Number Z26491 or a complement thereof under high stringency conditions.

Polymorphisms within the COMT gene can be detected by utilizing a number of techniques. Nucleic acid from any nucleated cell can be used as the starting point for such assay techniques, and may be isolated according to standard nucleic acid preparation procedures which are well known to those of skill in the art. Such techniques are explained fully in the literature. See, e.g., Current Protocols in Molecular Biology, eds. Ausubel et al. 1994 N.Y.: John Wiley & Sons; Molecular Cloning A Laboratory Manual, 2nd Ed., ed. by Sambrook, Fritsch and Maniatis 1989 Cold Spring Harbor Laboratory Press.

In an exemplary embodiment, there is provided a nucleic acid composition comprising a nucleic acid probe or primer including a region of nucleotide sequence which is capable of hybridizing to a sense or antisense sequence of a nucleic acid sequence for the human COMT gene set forth in GenBank Accession Number Z26491, or allelic variant, or 5′ or 3′ flanking sequences naturally associated with the subject COMT gene. The nucleic acid of a cell is rendered accessible for hybridization, the probe or primer is contacted with the nucleic acid of the sample, and the hybridization of the probe or primer to the sample nucleic acid is detected. Such techniques can be used to detect the presence of allelic variants at either the genomic or mRNA level.

A preferred detection method is allele specific hybridization using probes overlapping the polymorphic site and having about 5, 10, 20, 25, or 30 nucleotides around the polymorphic region. In a preferred embodiment of the invention, one or more probes capable of hybridizing specifically to allelic variants of the single nucleotide polymorphism within exon 4 of the human COMT gene are attached to a solid phase support, e.g., a “chip”. Oligonucleotides can be bound to a solid support by a variety of processes, including lithography. For example a chip can hold up to about 250,000 oligonucleotides. Mutation detection analysis using these chips comprising oligonucleotides, also termed “DNA probe arrays” is described e.g., in Cronin et al. 1996 Human Mutation 7:244. In one embodiment, a chip comprises both allelic variants of the polymorphic region of the COMT gene. The solid phase support is then contacted with a test nucleic acid and hybridization to the specific probes is detected. Accordingly, the identity of both allelic variants of the COMT gene can be identified in a simple hybridization experiment.

In certain embodiments, detection of the polymorphism comprises utilizing a primer in a polymerase chain reaction (PCR) (see, e.g. U.S. Pat. Nos. 4,683,195 and 4,683,202), such as anchor PCR or RACE PCR, or, alternatively, in a ligase chain reaction (LCR) (see, e.g., Landegran et al. 1988 Science 241:1077-1080; and Nakazawa et al. 1994 PNAS 91:360-364), the latter of which can be particularly useful for detecting polymorphisms in a gene (see Abravaya et al. 1995 Nucl Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to the COMT gene under conditions such that hybridization and amplification of the COMT allele (if present) occurs, and (iv) detecting the presence or absence of an amplification product, or detecting the size of the amplification product and comparing the length to a control sample. It is anticipated that PCR, LCR or any other amplification procedure (e.g., self sustained sequence replication: Guatelli, J. C. et al. 1990 PNAS USA 87:1874-1878; transcriptional amplification system: Kwoh, D. Y. et al. 1989 PNAS USA 86:1173-1177; or Q-Beta Replicase: Lizardi, P. M. et al. 1988 Bio/Technology 6:1197), may be used as a preliminary step to increase the amount of sample on which can be performed any of the techniques for detecting polymorphisms described herein.

In a preferred embodiment of the subject assay, allelic variants of the COMT gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531) can be used to score for the presence of specific polymorphisms by development or loss of a ribozyme cleavage site.

In yet another embodiment, any of a variety of sequencing reactions known in the art can be used to directly sequence the COMT gene and detect polymorphisms by comparing the sequence of the sample COMT with the known sequence. Exemplary sequencing reactions include those based on techniques developed by Maxim and Gilbert 1977 PNAS USA 74:560, or Sanger et al. PNAS USA 74:5463. It is also contemplated that any of a variety of automated sequencing procedures may be utilized when performing the subject assays (Biotechniques 1995 19:448), including sequencing by mass spectrometry (see, for example, PCT publication WO 94/16101; Cohen et al. 1996 Adv Chromatogr 36:127-162; and Griffin et al. 1993 Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. 1985 Science 230:1242). In general, the art technique of “mismatch cleavage” starts by providing heteroduplexes formed by hybridizing (labelled) RNA or DNA containing a control sequence with RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the presence of the polymorphism. See, for example, Cotton et al. 1988 PNAS USA 85:4397; Saleeba et al. 1992 Methods Enzymol 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting polymorphisms in COMT cDNAs obtained from samples of cells. For example, the mutY enzyme of E. coli cleaves A at G/A mismatches. According to an exemplary embodiment, a probe based on a reference sequence is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify the allelic variant of a polymorphic region in the COMT gene. For example, single strand conformation polymorphism (SSCP) may be used to detect differences in electrophoretic mobility between polymorphisms (Orita et al. 1989 PNAS USA 86:2766, see also Cotton 1993 Mutat Res 285:125-144; and Hayashi 1992 Genet Anal Tech Appl 9:73-79). Single-stranded DNA fragments of sample and control COMT nucleic acids are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. 1991 Trends Genet 7:5).

In yet another embodiment, the movement of polymorphic fragments in polyacrylamide gels containing a gradient of denaturant is assayed using denaturing gradient gel electrophoresis (DGGE) (Myers et al. 1985 Nature 313:495). When DGGE is used as the method of analysis, DNA will be modified to insure that it does not completely denature, for example, by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner 1987 Biophys Chem 265:12753).

Examples of other techniques for detecting the presence of the allelic variant of a polymorphic region include, but are not limited to, selective oligonucleotide hybridization, selective amplification, or selective primer extension. For example, oligonucleotide primers may be prepared in which the nucleotide difference in allelic variants is placed centrally and then hybridized to target DNA under conditions which permit hybridization only if a perfect match is found (Saiki et al. 1986 Nature 324:163; Saiki et al. 1989 PNAS USA 86:6230). Such allele specific oligonucleotide hybridization techniques may be used to test one polymorphic region per reaction when oligonucleotides are hybridized to PCR amplified target DNA or both polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the polymorphic region of interest in the center of the molecule (so that amplification depends on differential hybridization) (Gibbs et al. 1989 Nucleic Acids Res 17:2437-2448) or at the extreme 3′ end of one primer where, under appropriate conditions, mismatch can prevent, or reduce polymerase extension (Prossner 1993 Tibtech 11:238). In addition it may be desirable to introduce a novel restriction site in the region of the polymorphism to create cleavage-based detection (Gasparini et al. 1992 Mol Cell Probes 6:1). It is anticipated that in certain embodiments amplification may also be performed using Taq ligase for amplification (Barany 1991 PNAS USA 88:189). In such cases, ligation will occur only if there is a perfect match at the 3′ end of the 5′ sequence making it possible to detect the presence of a given polymorphism at a specific site by looking for the presence or absence of amplification. 100601 In another embodiment, identification of the allelic variant is carried out using an oligonucleotide ligation assay (OLA), as described, e.g., in U.S. Pat. No. 4,998,617 and in Landegren, U. et al. 1988 Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA in Nickerson, D. A. et al. 1990 PNAS USA 87:8923-8927. In this method, PCR is used to achieve the exponential amplification of target DNA, which is then detected using OLA.

Several techniques based on this OLA method have been developed and can be used to detect specific allelic variants of a polymorphic region of the COMT gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. 1996 Nucleic Acids Res 24:3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Several methods have been developed to facilitate the analysis of single nucleotide polymorphisms. In one embodiment of the invention, a single base polymorphism can be detected by using a specialized exonuclease-resistant nucleotide, as disclosed, e.g., in Mundy, C. R. (U.S. Pat. No.4,656,127). According to the method, a primer complementary to the allelic sequence immediately 3′to the polymorphic site is permitted to hybridize to a target molecule obtained from a particular subject. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO 91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO 91/02087) the method of Goelet, P. et al. is preferably a heterogeneous phase assay, in which the primer or the target molecule is immobilized to a solid phase.

Detection procedures may also be performed in situ directly upon tissue sections (fixed and/or frozen) of patient tissue obtained from biopsies or resections, such that no nucleic acid purification is necessary. Nucleic acid reagents may be used as probes or primers for such in situ procedures (see, for example, Nuovo, G. J. 1992 PCR in situ hybridization: protocols and applications, Raven Press, N.Y.).

Where a biological sample includes a COMT protein, the presence or absence of an amino acid may be detected using conventional means, e.g., an antibody which is specific for a variant sequence. For example, by using immunogens derived from a COMT protein, e.g., based on the cDNA sequences, anti-protein/anti-peptide antisera or monoclonal antibodies can be made by standard protocols (see, for example, Antibodies: A Laboratory Manual 1988 ed. by Harlow and Lane Cold Spring Harbor Press). A mammal, such as a mouse, a hamster or rabbit can be immunized with an immunogenic form of the peptide (e.g., a COMT protein or an antigenic fragment which is capable of eliciting an antibody response). Techniques for conferring immunogenicity on a protein or peptide include conjugation to carriers or other techniques well known in the art. An immunogenic portion of a COMT protein can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a COMT protein, anti-COMT antisera can be obtained and, if desired, polyclonal anti-COMT antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique originally developed by Kohler and Milstein 1975 Nature 256:495-497, the human B cell hybridoma technique (Kozbar et al. 1983 Immunology Today 4:72), and the EBV-hybridoma technique to produce human monoclonal antibodies (Cole et al. 1985 Monoclonal Antibodies and Cancer Therapy, Alan R. Liss, Inc. pp. 77-96). Hybridoma cells can be screened immunochemically for production of antibodies specifically reactive with a COMT protein of the present invention and monoclonal antibodies isolated from a culture comprising such hybridoma cells.

The term antibody as used herein is intended to include fragments thereof which are also specifically reactive with the subject human COMT proteins. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a COMT protein conferred by at least one CDR region of the antibody. In preferred embodiments, the antibody further comprises a label attached thereto and able to be detected, (e.g., the label can be a radioisotope, fluorescent compound, enzyme or enzyme co-factor).

Antibodies directed against COMT proteins may also be used to detect allelic variants. Such antibodies detect differences in the structure of a COMT protein. Structural differences may include, for example, differences in the size, electronegativity, or antigenicity of a COMT protein relative to an allelic variant. Protein from the tissue or cell type to be analyzed may easily be detected or isolated using techniques which are well known to one of skill in the art, including but not limited to Western blot analysis. For a detailed explanation of methods for carrying out Western blot analysis, see Molecular Cloning A Laboratory Manual 2nd ed. 1989 ed. by Sambrook, Fritsch and Maniatis, Cold Spring Harbor Laboratory Press. The protein detection and isolation methods employed herein may also be such as those described in Antibodies: A Laboratory Manual 1988 ed. by Harlow and Lane Cold Spring Harbor Press.

This can be accomplished, for example, by immunofluorescence techniques employing a fluorescently labeled antibody coupled with light microscopic, flow cytometric, or fluorimetric detection. The antibodies (or fragments thereof) useful in the present invention may, additionally, be employed histologically, as in immunofluorescence or immunoelectron microscopy, for in situ detection of COMT proteins. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine the presence of the COMT protein. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Those skilled in the art will know many other suitable carriers for binding antibody or antigen, or will be able to ascertain the same by use of routine experimentation.

One means for labeling an anti-COMT protein specific antibody is via linkage to an enzyme and use in an enzyme immunoassay (EIA) (Voller 1978 Diagnostic Horizons 2:1-7, 1978, Microbiological Associates Quarterly Publication, Walkersville, Md.; Voller, et al. 1978 J Clin Pathol 31:507-520; Butler 1981 Meth Enzymol 73:482-523; Maggio, ed. 1980 Enzyme Immunoassay, CRC Press, Boca Raton, Fla.; Ishikawa, et al. eds. 1981 Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect COMT proteins through the use of a radioimmunoassay (RIA) (see, for example, Weintraub, B. 1986 Principles of Radioimmunoassays, Seventh Training Course on Radioligand Assay Techniques, The Endocrine Society). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Alternatively, other techniques can be used to detect the variant sequences, including chromatographic methods, such as SDS PAGE, isoelectric focusing, HPLC, and capillary electrophoresis.

Additionally, an activity assay, such as described by Weinshilboum and Raymond, 1977 Am J Med Genet 29:125-135, can be utilized to determine the level of COMT activity.

Any cell type or tissue may be utilized in the detection procedures described above. In a preferred embodiment a bodily fluid, e.g., blood, is obtained from the subject to determine the presence of the allelic variant of a polymorphic region in the COMT gene. A bodily fluid, e.g., blood, can be obtained by known techniques (e.g., venipuncture). Alternatively, nucleic acid tests can be performed on dry samples (e.g., skin). For prenatal testing, fetal nucleic acid samples can be obtained from maternal blood as described in International Patent Application No. WO 91/07660 to Bianchi. Alternatively, amniocytes or chorionic villi may be obtained for performing prenatal testing.

When using RNA or protein to determine the presence of a specific allelic variant of a polymorphic region in the COMT gene, the cells or tissues that may be utilized must express the COMT gene. Preferred cells for use in these methods include erythrocytes. Alternative cells or tissues that express the COMT gene include liver, kidneys, gastrointestinal tract, spleen, submaxillary glands, pancreas, lung, eye, spinal membranes, skin, breast, uterus, ovaries, and brain.

Frontal Lobe Tests

The tests that can be administered to examine frontal lobe cognitive function include but are not limited to N-back, Wisconsin Card Sort Test, Trails B, and Verbal Fluency. These tests are described in Fleming, K. et al. “Applying working memory constructs to schizophrenic cognitive impairment” In: David A. S. and Cutting J. C. (Eds.) 1994 The Neuropsychology of Schizophrenia. Hillsdale, N.J.: Erlbaum

The N-back task is described in Callicott, J. H. et al. 1999 Cerebral Cortex 9:20-26.

The Wisconsin Card Sort Test is described in Berman, K. F. et al. 1995 Neuropsychologia 33: 1027-1046.

The Trails B test is described in Goldberg, T. E. et al. 1988 Int J Neurosci 42:51-58, and Lezak, M. 1995 Neuropsychological Assessment Oxford, N.Y.

The Verbal Fluency test is described in Gourovitch, M. L. et al. 1996 Neuropsychology 6:573-577, and Lezak, M. 1995 Neuropsychological Assessment Oxford, N.Y.

Kits

The invention further provides kits for use in the methods described herein. For example, the kit can comprise at least one probe nucleic acid, a primer set, or antibody reagent described herein, which may be conveniently used, e.g., in clinical or laboratory settings. The kit can further comprise instructions for using the kit.

COMT Inhibitors

Suitable COMT-inhibitors and methods for preparation thereof have been described, e.g., in GB 2200109, EP 237929 and PCT application PCT/FI96/00295, and may be developed. COMT inhibitors that penetrate the blood brain barrier are preferred.

The invention is directed to a method for the manufacture of a medicament for use in the prevention or treatment of a human condition that involves deficits in prefrontal cognitive function by combining a COMT inhibitor or its pharmaceutically acceptable salt or ester in admixture with a physiologically acceptable excipient for administration to an individual in need thereof. In some embodiments, the human condition is a member selected from the group consisting of Parkinson's Disease, AIDS, normal aging, brain injury, alcoholism, schizophrenia, depression, obsessive-compulsive disorder, attention deficit hyperactivity disorder, autism, impulse control disorder, addiction, Alzheimer's disease and other forms of dementia, mental retardation, and normal cognition. In other embodiments, the individual has impaired prefrontal cognitive function as detected by any of the herein-described methods, or may be a normal subject.

The invention is also directed to a method for the prevention or treatment of a human condition that involves deficits in prefrontal cognitive function by administering to an individual in need thereof an effective amount of a COMT inhibitor or its pharmaceutically acceptable salt or ester to prevent or treat said condition. In some embodiments, the human condition is a member selected from the group consisting of Parkinson's Disease, AIDS, normal aging, brain injury, alcoholism, schizophrenia, depression, obsessive-compulsive disorder, attention deficit hyperactivity disorder, autism, impulse control disorder, addiction, Alzheimer's disease and other forms of dementia, mental retardation, and normal cognition. In other embodiments, the individual has impaired prefrontal cognitive function as detected by any of the herein-described methods, or may be a normal subject.

In a variation, the invention is directed to a method for the manufacture of a medicament for use in improving prefrontal cognitive function in a normal subject comprising combining a COMT inhibitor or its pharmaceutically acceptable salt or ester in admixture with a physiologically acceptable excipient for administration to the normal subject.

In another variation, the invention is directed to a method of improving prefrontal cognitive function in a normal subject comprising administering to the normal subject a COMT inhibitor or its pharmaceutically acceptable salt or ester.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀ Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable excipients. Thus, the compounds and their physiologically acceptable salts and solvates may be formulated for administration by, for example, injection, inhalation or insufflation (either through the mouth or the nose) or oral, buccal, parenteral or rectal administration, or through molecular techniques using gene therapy.

For such therapy, the compounds of the invention can be formulated for a variety of loads of administration, including systemic and topical or localized administration. Techniques and formulations generally may be found in Reminington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time. This technology utilizes microspheres of precapillary size which can be injected via a catheter into any selected part of the e.g. brain or other organs. The administered therapeutic is slowly released from these microspheres and taken up by surrounding tissue cells.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compounds of the invention are formulated into ointments, salves, gels, or creams as generally known in the art.

In clinical settings, a gene delivery system for a COMT nucleotide sequence encoding an antisense, ribozyme or dominant negative mutant can be introduced into a patient by any of a number of methods, each of which is familiar in the art. For instance, a pharmaceutical preparation of the gene delivery system can be introduced systemically, e.g., by intravenous injection, and specific transduction of the gene in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the gene is more limited with introduction into the individual being quite localized. For example, the gene delivery vehicle can be introduced by catheter (see U.S. Pat. No. 5,328,470) or by stereotactic injection (e.g., Chen et al. 1994 PNAS USA 91:3054-3057).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### EXAMPLE 1

Subjects and Cognitive Testing

Subjects were recruited from local and national sources as volunteers for the “CBDB/NIMH sibling study”, as previously described (Egan, M. et al. 2000 Am J Psychiatry 157:1309-1316). Briefly, all participants gave written informed consent of an IRB approved protocol. Most families had two eligible full siblings (at least one of whom met DSM-IV criteria for schizophrenia or schizoaffective disorder, depressed subtype). All subjects had to be from 18 to 60 years of age, above 70 in premorbid IQ, and able to give informed consent. Applicants with significant medical problems, history of head trauma, alcohol or drug abuse within the last six months were excluded. All subjects were medically screened and interviewed by a research psychiatrist using the Structured Clinical Interview (SCID) (First, M. B. et al. 1996 User's Guide for the SCID-I for DSM-IV Axis I Disorders-Research Version Biometrics Research, New York).

To reduce the possibility of artifactual association due to ethnic stratification, the final sample included only individuals of European ancestry born and educated in the U.S. This sample included 175 patients with schizophrenia, 219 healthy siblings, and 55 control subjects.

Subjects performed the Wisconsin Card Sorting Test (WCST). “Perseverative errors” was used as a dependent measure because it is thought to best reflect prefrontal function. Scores were transformed to t scores and normalized for age and education based on population means, a routine convention (Heaton, R. K. et al. 1993 Wisconsin Card Sorting Test Manual Psychological Assessment Resources, Inc. Odessa, Fla.). Thus, better performance is reflected in a higher t score. IQ (from the Wechsler Adult Intelligence Scale, revised edition, or WAIS-R) and reading comprehension (using the WRAT, a measure of premorbid IQ) were also collected (Jastak, S. & Wilkinson, G. S. 1984 Wide Range Achievement Test Jastak Associates, Wilmington, Del.).

Neuroimaging

Two cohorts of siblings (all nonsmokers) and one cohort of probands were randomly selected, based on scanner availability. Blood oxygen level dependent (BOLD) fMRI was performed while subjects took the 2-back and zero-back versions of the N-back task (Callicott, J. H. et al. 2000 Cereb Cortex 10:1078-1092). In contrast to the WCST, the N-back is a relatively simple working memory task more suitable for fMRI.

The N-back task was presented via a fiber-optic goggle system and responses recorded via a pneumatic button box. Stimuli were displayed randomly at a rate of 1.8 per sec. All subjects were first trained to maximal performance. The first group of unaffected siblings (n=16) and the group of patients with schizophrenia (n=11) were studied with an echo planar imaging (EPI) BOLD fMRI sequence at 1.5 Tesla (Callicott, J. H. et al. 2000 Cereb Cortex 10:1078-1092). The second sibling group (n=11) was studied using a more rapid scanning pulse sequence, fast spiral imaging also at 1.5 Tesla (Yang, Y. et al. 1996 Magn Reson Med 36:620-6).

Whole brain EPI data were collected in a modified block design with pseudo-randomized intermixing of zero-back and 2-back working memory tasks. Fast spiral imaging data were collected using a simple block design alternating between zero-back and 2-back (16 sec/task epoch) occurring during one 256 second run. All fMRI data were reconstructed, registered, linear detrended, globally normalized, and then smoothed (10 mm Gaussian kernel) prior to analysis within Statistical Parametric Mapping (SPM) (Friston, J. K. et al. 1995 Human Brain Mapping 2:189-210). All data were rigorously screened for artifacts as previously described (Callicott, J. H. et al. 2000 Cereb Cortex 10:1078-1092). Individual data from 18 task epochs were collapsed as adjusted means and then entered into a general linear model within SPM96 (for cohort 1) or SPM 99 (for cohort 2) (Wellcome Department of Cognitive Neurology, London). We first estimated parameters that reflected activation as a contrast between the two-back task and the zero-back task. These parameter estimates were then entered into a second analysis to test inferences about differential activations among the three genotype groups. This is formally identical to a random effects analysis where the subject effect is a random effect. Because we had an anatomically specified hypothesis about prefrontal activation, we used an uncorrected threshold of p=0.005 (voxelwise) to identify these regionally specific differences. The resultant statistical maps were then rendered onto a 3-D standard brain.

Genetic Analysis

Blood was collected from all subjects as well as all available parents of patients with schizophrenia. DNA was extracted using standard methods. DNA from 104 pairs of parents were available for the final analysis. COMT Val^{108/158}Met genotype was determined as a restriction fragment length polymorphism following PCR amplification and digestion with NlaIII, similar to a previously described method (Lachman, H. M. et al. 1996 Pharmacogenetics 6:243-50).

Briefly, a 109 base pair polymerase chain reaction (PCR) product was generated in 30 cycles with annealing temperature of 54° C. using the following primers: Comt1 nt 1881 5′CTCATCACCATCGAGATCAA (SEQ ID NO: 5); and Comt2 nt 1989 5′CCAGGTCTGACAACGGGTCA (SEQ ID NO: 6).

Alleles for Val and Met were discriminated by digesting the PCR product with NlaIII at 37° C. for 4 hrs, followed by 4.5% agarose gel electrophoresis. The Val/Val homozygote (86 and 23 bp), Met/Met homozygote (68, 23 and 18 bp) and the Val/Met heterozygote (86, 68, 23 and 18 bp) were visualized by ethidium bromide staining.

To address at a genomic level the issue of potential population admixture, nineteen unlinked, short tandem repeat (STR) markers, all with heterozygosities >65%, were genotyped using PCR and gel analysis as previously described (Straub, R. E. et al. 1993 Genomics 15:48-56) in selected subjects. The markers were: D1S1612, D1S1678, D2S1356, D4S1280, D5S1471, D6S1006, D7S2847, D17S1308, D18S843, D18S535, D19S714, D20S604, D20S477, D20S481, D21S1437, D21S1446, D22S445, SLC6A3 3′UTR VNTR (GenBank accession no. 162767), and the (TAA) repeat in locus HSMHC3A5 (GenBank accession no. U89335).

Statistical Analyses

Between groups comparisons of demographic data were performed using paired or unpaired t-tests or Chi-square, as appropriate. To avoid lack of independence among family members, we used one randomly selected sibling per family for comparisons with the control group. The effects of COMT genotype were analyzed several ways. First, groups were compared using standard parametric techniques (case/control comparisons). Second, to avoid spurious results due to admixture, we used transmission disequilibrium tests (TDT) (Spielman, R. S. et al. 1993 Am J Hum Genet 52:506-16; Allison, D. B. et al. 1999 Am J Hum Genet 64:1754-63), which are family based methods that sacrifice power substantially.

The effect of COMT genotype on WCST performance was assessed using two case/control analyses: 1) analysis of variance (ANOVA) and 2) multiple regression. With ANOVA, we first included all subjects. Since this assumes independence of individuals, we also report ANOVA results including only patients and controls. Second, using multiple regression we tested the hypothesis that the number of Met alleles was parametrically related to enhanced performance (patients and controls only); diagnostic group was included as the only additional independent variable. Next, we performed a family based test to examine the effect of COMT genotype on WCST performance, quantitative sib transmission disequilibrium test (TDT) (Allison, D. B. et al. 1999 Am J Hum Genet 64:1754-63). Subsequently, we examined whether admixture was present in Val/Val and Met/Met groups for patients and controls (FIG. 1) by comparing allele frequencies of 19 unlinked polymorphic genetic markers using an overall χₛ² as described by Pritchard and Rosenberg (Pritchard, J. K. & Rosenberg, N. A. 1999 Am J Hum Genet 65:220-8).

The effect of COMT genotype on risk for schizophrenia was analyzed using both case control and family based methods. The case/control analysis was a comparison of allele frequencies. The family based analysis used the TDT (Spielman, R. S. et al. 1993 Am J Hum Genet 52:506-16). A critical issue in assessing the significance of association with phenotypic measures is the likelihood of type I errors. Many genes and phenotypes can be evaluated for schizophrenia and may ultimately be examined in this dataset, but Bonferroni correction for all possible combinations that may ultimately be performed seems overly stringent. The approach here was to selectively analyze a single candidate functional polymorphism, chosen for its biological effect, against a target phenotype likely impacted by this biological effect.

### EXAMPLE 2

A functional polymorphism in the gene for catechol-O-methyltransferase (COMT) has been shown to affect executive cognition and the physiology of the prefrontal cortex in humans, probably by changing prefrontal dopamine flux. The COMT valine allele, associated with relatively poor prefrontal function, is also a gene that increases risk for schizophrenia. While poor performance on executive cognitive tasks and abnormal prefrontal function are characteristics of schizophrenia, so is psychosis, which has been related to excessive subcortical presynaptic dopamine activity. Studies in animals have shown that diminished prefrontal dopamine neurotransmission leads to upregulation of subcortical dopamine activity. We measured tyrosine hydroxylase (TH) mRNA in mesencephalic dopamine neurons in human brain and found that the COMT valine allele is also associated with increased TH gene expression. This indicates that COMT genotype is a heritable aspect of dopamine (DA) regulation and it further explicates the mechanism by which the COMT valine allele increases susceptibility for schizophrenia.

Dopamine (DA) neurotransmission has been shown in both human and non-human primates to be critical for cognitive functions subserved by the prefrontal cortex (PFC), such as executive cognition and working memory (Sawaguchi, T. & Goldman-Rakic, P. S. 1994 J Neurophysiol 71:515-528; Williams, G. V. & Goldman-Rakic P. S. 1995 Nature 376:572-575). DA levels in the PFC are determined by DA biosynthesis and release and by the rate of re-uptake and degradation. Breakdown may be particularly relevant to DA inactivation in the PFC in view of recent evidence that the DA transporter is rarely expressed within synapses in this region (Lewis, D. A. et al. 2001 J Comp Neurol 432:119-136). COMT is an important enzyme involved in the breakdown of DA and converts DA to 3-methoxytyramine (3-MT) and the DA metabolite dihydroxyphenylacetic acid (DOPAC) to homovanilic acid (HVA) (Boulton, A. A., Eisenhofer G. 1998 Adv Pharmacol 42:273-292).

The human COMT gene contains a common functional polymorphism—a valine (Val)/methionine (Met) substitution—at amino acid 108/158. The Met allele results in a heat-labile protein with a four-fold reduction in enzymatic activity (Mannisto, P. T. & Kaakkola, S. 1999 Pharmacol Rev 51:593-628). Studies in peripheral blood and in liver indicate that this functional polymorphism accounts for most of the variation in peripheral COMT activity between individuals. Therefore, COMT genotype might also contribute to differences in prefrontal function between individuals. Consistent with this prediction, we found in a large sample of subjects (n=465) that COMT genotype is associated with variations in executive cognition and with PFC physiological activity during working memory. As expected, Met/Met individuals had the best performance on executive cognition tasks, Val/Val individuals had the worst, and Val/Met individuals were intermediate. These findings, in conjunction with the specific effect of COMT on PFC DA flux and on memory found in COMT knockout mice (Gogos, J. A. et al. 1998 PNAS USA 95:9991-9996; Kneavel, M. et al. 2000 Society for Neuroscience 30th Annual Meeting, New Orleans, 571.20 abstr.; Huotari, M. et al. 2002 Eur J Neurosci 15:246-256), support the notion that COMT genotype affects DA neurotransmission in the PFC.

In addition to its role in normal cognition, DA neurotransmission in PFC has been implicated in the pathophysiology of schizophrenia (Weinberger, D. R 1987 Arch Gen Psychiatry 44:660-669; Akil, M. et al. 1999 Am J Psychiatry 156:1580-1589). Patients with schizophrenia exhibit deficits in cognitive tasks that are dependent upon the function of the PFC (Weinberger, D. R. et al. 1986 Arch Gen Psychiatry 43:114-124; Carter, C. S. et al. 1998 Am J Psychiatry 155:1285-1287), and show abnormalities of prefrontal physiology during performance of such tasks (Weinberger, D. R. et al. 1986 Arch Gen Psychiatry 43:114-124; Weinberger, D. R. et al. 1988 Arch Gen Psychiatry 45:609-615; Carter, C. S. et al. 1998 Am J Psychiatry 155:1285-1287; Callicott, J. H. et al. 2000 Cereb Cortex 10:1078-1092; Barch, D. M. et al. 2001 Arch Gen Psychiatry 58:280-288). Moreover, these functional abnormalities have been related to measures of cortical dopamine activity in vivo (Weinberger, D. R. et al. 1988 Arch Gen Psychiatry 45:609-615; Daniel, D. G. et al. 1991 J Neurosci 11:1907-1917; Okubo, Y. et al. 1997 Nature 385:634-636), and evidence of abnormal dopaminergic innervation of PFC has been found in postmortem brains of patients with schizophrenia (Akil, M. et al. 1999 Am J Psychiatry 156:1580-1589). Consistent with this evidence, inheritance of the Val allele has been found in family-based association studies to slightly increase risk for schizophrenia, as described herein and in (Li, T. et al. 1996 Psychiatr Genet 6:131-133; Kunugi, H. et al. 1997 Psychiatr Genet 7:97-101; De Chaldee, M. et al. 1999 Am J Med Genet 88:452-457), implicating COMT as a susceptibility gene for schizophrenia.

The mechanism by which inheritance of the COMT Val allele increases risk for schizophrenia may be related to its adverse impact on prefrontal DA signaling and prefrontal function. However, DA neurotransmission in PFC has also been shown to affect subcortical DA activity, which is implicated in both the psychotic symptoms of schizophrenia (Carlsson, A. 1995 Int Clin Psychopharmacol 10 Suppl 3:21-28; Grace, A. 2000 Brain Res Rev 31:330-341; Laruelle, M. 2000 Brain Res Brain Res Rev 31:371-384) and the therapeutic response to anti-dopaminergic drugs (Deutch, A. Y. 1993 J Neural Transm Gen Sect 91:197-221; Kinon, B. J. & Lieberman, J. A. 1996 Psychopharmacology (Berl) 124:2-34). DA flux in PFC modulates the activity of excitatory cortical neurons that project both directly and indirectly to mesencephalic DA neurons (Haber, S. N. & Fudge, J. L. 1997 Crit Rev Neurobiol 11:323-342; Lu, X. Y. et al. 1997 Synapse 25:205-214; Carr, D. B. & Sesack, S. R. 2000 J Neurosci 20:3864-3873). Under experimental conditions in animals, reduced DA signaling in the PFC leads to increased responsivity of midbrain DA neurons to stimuli such as stress (Kolachana, B S. et al. 1995 Neuroscience 69:859-868; Harden, D. G. et al. 1998 Brain Res 794:96-102). The notion that overactive mesencephalic dopamine neurons might be a “downstream” effect of an abnormality in prefrontal function has been proposed as an explanation for the coexistence of both cortical and subcortical dopaminergic abnormalities in schizophrenia (Weinberger, D. R 1987 Arch Gen Psychiatry 44:660-669; Daniel, D. G. et al. 1991 J Neurosci 11:1907-1917; Grace, A. 2000 Brain Res Rev 31:330-341). Therefore, to the extent that COMT genotype affects prefrontal function, it may contribute to risk for schizophrenia not only because of its biological effects at the level of PFC, but also because of indirect effects mediated by cortical neurons projecting to brainstem DA neurons.

We hypothesized that the relatively selective effect of COMT at the PFC level would alter the homeostasis of mesencephalic DA neurons in the normal human brain in a predictable direction. Thus, in comparison with the Met allele, the Val allele, which is likely associated with relatively diminished prefrontal DA signaling, would result in increased recruitment of mesencephalic DA activity. To test this hypothesis, we compared mRNA levels of tyrosine hydroxylase (TH), the rate-limiting enzyme for dopamine biosynthesis, in dopamine neurons of postmortem human brain specimens from normal subjects with the Val/Val genotype and those with the Val/Met genotype.

Characteristics of Subjects

Human brain specimens were obtained, during the course of routine autopsy, through the Office of the Medical Examiners of the District of Columbia with the informed consent of the next of kin. Twenty-three normal controls were included in this study. Ten cases with Val/Val genotype (7 males and 3 females) were compared to 13 cases of Val/Met genotypes (8 males and 5 females). All Cases in the Val/Val group were African American compared to 10 African Americans and 3 Caucasians in the Val/Met group. Mean PMI is comparable between the 2 groups (31.0±15.5 for Val/Val and 33.8±17.5 for Val/Met), as is mean age (40.9±14.9 for Val/Val and 49.9±7.5 for Val/Met) and mean pH (6.42±0.23 for Val/Val and 6.39±022 for Val/Met). Clinical records were reviewed by two board-certified psychiatrists and collateral information was obtained, whenever possible, from telephone interviews with surviving relatives of the deceased. Blood and/or brain toxicology screens were obtained in each case. We excluded subjects with known history of neurological disorders, psychiatric disorders or substance abuse and all cases with prolonged agonal state. Each case was examined macroscopically and microscopically by an experienced neuropathologist. We excluded cases with significant neuropathological abnormalities or that met criteria for Alzheimer's disease. The collection of human brain specimens was approved by the Institutional Review Board of the NIMH intramural Research Program.

Tissue Specimens

In each case, the midbrain was cut into 1-2 cm blocks in a plane perpendicular to its long axis. Tissue blocks were frozen immediately in a mixture of dry ice and isopentane, cryostat sectioned at 14 μm, thaw-mounted onto poly-lysine-subbed microscope slides, then dried and stored at −80° C. Every 50^{th} section was stained for Nissl substance with thionin. Anatomical levels corresponding to FIG. 57 in the “Atlas of the Human Brainstem” by Paxinos and Huang (Paxinos, G. 1995 Atlas of the Human Brainstem. San Diego: Academic Press) were identified in each case using Nissl-stained sections and TH immunocytochemistry. Regional boundaries of the DA cell groups within the midbrain were determined according to McRitchie (McRitchie, D. & Halliday, G. 1995 Neuroscience 65:87-91). Five cell groups were identified; the substantia nigra pars lateralis (SNL), the dorsal and ventral tiers of the pars compacta (SND and SNV respectively), the paranigral nucleus (PN) and the ventral tegmental area (VTA). These nuclei were chosen because they could be reliably identified in this anatomical level. pH measures were conducted in each case using 500 mg of tissue homogenate from the cerebellum and a 420A Orion pH meter with Orion's highly sensitive glass pH SURE-FLOW electrode.

In Situ Hybridization

Tissue sections 14 Jim thick were hybridized with ³⁵S-labeled riboprobes for TH, for the dopamine transporter (DAT) and for cyclophilin. We used a 545 bp long riboprobe for human TH (Uhl, G R. et al. 1994 Ann Neurol 35:494-498) and a 350 bp riboprobe for human DAT (Joh, T H. et al. 1998 Adv Pharmacol 42:33-36). In situ hybridization for TH and DAT was performed as previously described (Little, K. et al. 1998 Arch Gen Psychiatry 55:793-799). Two to four tissue sections from each subject at the chosen anatomical level were included. To control for between-experiment variations, tissue sections from all subjects were always processed in the same experiment. Cyclophilin in situ hybridization was conducted using ³⁵S-labeled riboprobe for human cyclophilin 103 bp cDNA from exons 1 and 2 of the human gene (Ambion, Austin Tex.) and the Whitfield method (Whitfield, H. J. et al. 1990 Cell Mol Neurobiol 10:145-157). Following overnight hybridization at 60° C. in humidified chambers, slides were placed in X-ray cassettes along with ¹⁴C standards (American Radiolabeled Chemicals Inc., St. Louis, Mo.) and apposed to Kodak BioMax MR autoradiographic film for 4-20 days.

Quantitative Analysis

Hybridization was quantified by measuring the optical density of the X-ray film with NIH Image software version v.1.61. All quantitative analyses were conducted blind to genotype. We sampled five DA cell groups from each side (SNPL, SNPD, SNPV, PN and the VTA). An area of 1.76 mm² in each cell group was sampled selecting the region of highest density. Thus, from 20-40 measurements were taken from each case for TH and for DAT. The results from all sections and both sides were averaged to produce five mean measures (one for each cell group) per subject. Mean values from each cell group and a total (sum) of means of all five were used for statistical analyses. The data were analyzed using COMT genotype (2) by cell group (5) analyses of variance on optical density measures of mean mRNA levels. In addition, analyses of covariance including age, gender, pH and PMI as individual co-variates were also conducted. Post hoc analyses were performed when appropriate using the Tukey HSD test.

Genetic Analysis

Frozen tissue samples were collected from the cerebellum of all cases. DNA was extracted using standard methods. COMT Val^{108/158}Met genotype was determined as a restriction fragment length polymorphism following PCR amplification and digestion with N1aIII as described above. Of twenty four brains originally genotyped, only one had a Met/Met genotype, which is not surprising considering the allele frequencies of the study population (Palmatier, M. A. et al. 1999 Biol Psychiatry 46:557-567). This brain was excluded from further analysis.

### COMT Genotype and Dopamine Regulation in the Human Brain

We found a main effect of COMT genotype on TH mRNA levels expressed as a summed measure in all five of the mesencephalic DA cell groups (F=5.63, df=1, 22, p=0.02, effect size=0.9 d) and as predicted, Val/Val cases had significantly greater expression than Val/Met cases (41.8 % increase).

In an analysis of individual cell groups, significant main effects of genotype were found in the SNV (71.6% difference, F=16.6, df=1, 22, p=0.0005) and the SND (47.6% increase, F=4.51, df=1, 22, p=0.04). None of the other cell groups showed significant differences and co-varying for factors such as age, gender, pH or postmortem interval did not affect these results (all F<2.18, all p>0.15).

TH gene expression has been shown to be dependent on the activity of dopamine neurons (Nagatsu, T. 1995 Essays Biochem 30:15-35; Kumer, S. C. & Vrana K. E 1996 J Neurochem 67:443-462; Tank, A. W. et al. 1998 Adv Pharmacol 42:25-29), thus, the difference in TH mRNA levels between the two COMT genotypes presumably reflects a difference in the activity of DA neurons. To further test the specificity of this finding, we examined expression of DAT mRNA, also expressed in DA neurons but not in an activity dependent manner (Hoffman, B. J. et al. 1998 Front Neuroendocrinol 19:187-231; Heinz, A. et al. 1999 Synapse 32:71-79), and cyclophilin, a constitutively expressed protein unrelated to DA metabolism. There were no effects of COMT genotype on the expression of either of these genes. The lack of effect of COMT genotype on DAT or cyclophilin mRNA, in contrast to TH, is presumably because TH mRNA levels reflect the activity of DA neurons while the others do not. Differential modification of DAT and TH mRNA levels in the human mesencephalon has also been reported in Parkinson's disease and Alzheimer's disease (Joyce, J. N. et al. 1997 Mov Disord 12:885-897).

Header

We have found that COMT Val¹⁵⁸/Met genotype affects TH gene expression in mesenchephalic DA neurons, and presumably the dopaminergic function of these neurons. COMT is found in low abundance in DA neurons and may only be expressed in specific subpopulations (Lundstrom, K. et al. 1995 Biochim Biophys Acta 1251:1-10). Kastner et al. (Kastner, A. et al. 1994 Neuroscience 62:449-457) found that DA neurons in the VTA and the SNL in the human were weakly immunoreactive for COMT protein, while DA neurons in the other cell groups of the mesostriatal system show no COMT expression. In contrast, COMT is expressed in striatal and cortical neurons that receive DA projections. Taken together, these findings suggest that the degradation of DA at most DA synapses involves postsynaptic rather than presynaptic COMT. Therefore, at least part of the observed relationship between COMT genotype and TH gene expression is likely to be mediated by the effect of COMT activity in cell populations other than DA neurons.

Several lines of evidence suggest that DA signaling in the PFC may mediate the effects of COMT genotype on mesencephalic DA function. First, in COMT knockout mice, DA levels in the striatum are not altered, but DA levels in the PFC are increased, and heterozygote knockouts are intermediate between homozygote knockouts and wild type animals (Gogos, J. A. et al. 1998 PNAS USA 95:9991-9996; Huotari, M. et al. 2002 Eur J Neurosci 15:246-256). Importantly, changes in norepinephrine levels are not found in these animals. Second, the PFC projects to the mesencephalon in both rodents and primates (Haber, S. N. & Fudge, J. L. 1997 Crit Rev Neurobiol 11:323-342; Lu, X. Y. et al. 1997 Synapse 25:205-214; Carr, D. B. & Sesack, S. R. 2000 J Neurosci 20:3864-3873) and burst firing of DA neurons depends upon the integrity and activity of these glutamatergic projections (Svensson, T. H. & Tung, C. S. 1989 Acta Physiol Scand 136:135-136; Murase, S. et al. 1993 Neurosci Lett 157:53-56; Karreman, M. & Moghaddam B. 1996 J Neurochem 66:589-598). Some of the pyramidal neurons contacted by DA afferents in the PFC project to DA neurons in the midbrain, both monosynaptically and polysynaptically through GABA intermediaries (Carr, D. B. & Sesack, S. R. 2000 J Neurosci 20:3864-3873). Third, 6-hydroxy-DA lesions of the PFC, which destroy DA terminals, have been shown to alter baseline firing of mesencephalic DA neurons and their response to stress (Harden, D. G. et al. 1998 Brain Res 794:96-102) and DA blockade in PFC leads to increased release of DA in striatal terminals (Roberts, A C. et al. 1994 J Neurosci 14:2531-2544; Kolachana, B S. et al. 1995 Neuroscience 69:859-868). Finally, levels of N-acetyl aspartate, an intracellular marker of neuronal integrity, assayed in vivo within PFC with proton NMR spectroscopy, have been shown to correlate inversely with amphetamine-stimulated striatal DA function in humans (Bertolino, A. et al. 2000 Neuropsychopharmacoly 22:125-132). Although regions other than the PFC, such as the hippocampus, also affect subcortical DA (Floresco, S. B. et al. 2001 J Neurosci 21:4915-4922), PFC is the only region known to project directly to DA neurons (Carr, D. B. & Sesack, S. R. 2000 J Neurosci 20:3864-3873). Moreover, in clinical studies, only PFC N-acetyl-aspartate (NAA) levels predicted the responsiveness of subcortical DA to amphetamine challenge (Bertolino, A. et al. 2000 Neuropsychopharmacoly 22:125-132). These convergent findings implicate PFC projections to mesencephalic DA neurons in the effects of COMT genotype on TH gene expression.

It is of interest that we observed the greatest genotype effects on TH mRNA levels in ventral tier of the SN and no effects in the VTA, the PN or the SNL. The ventral tier in the primate projects primarily to the striatum and amygdala (Haber, S. N. & Fudge, J. L. 1997 Crit Rev Neurobiol 11:323-342). In a study of COMT protein expression (Kastner, A. et al. 1994 Neuroscience 62:449-457), ventral tier neurons were unique in that they did not express COMT protein. Thus, our results suggest that the effects of COMT genotype on TH regulation are greatest in cell groups that do not express COMT and that do not project back to the PFC. These cell group specific effects appear to be consistent with anatomical data in animals that prefrontal inputs to striatal-projecting DA neurons are through GABA intermediates (Carr, D. B. & Sesack, S. R. 2000 J Neurosci 20:3864-3873).

Our finding that COMT genotype is a heritable factor in DA modulation may further clarify the mechanism by which the COMT Val allele increases risk for schizophrenia and possibly other psychotic states. Several lines of evidence suggest that a cortical hypo-dopaminergic state is accompanied by a subcortical hyper-dopaminergic state in schizophrenia, thereby contributing to cognitive and psychotic symptoms, respectively (Weinberger, D. R 1987 Arch Gen Psychiatry 44:660-669; Grace, A. 2000 Brain Res Rev 31:330-341). Such reciprocal relationships between cortical and subcortical DA systems has been demonstrated repeatedly in animal experiments (Pycock, C. et al. 1980 Nature 286:74-76; Finlay, J. M. & Zigmond M. J. 1997 Neurochem Res 22:1387-1394; Lipska, B. K. & Weinberger, D. R. 1998 Adv Pharmacol 42:806-809; Harden, D. G. et al. 1998 Brain Res 794:96-102). Increased responsivity of striatal dopaminergic terminals to amphetamine in schizophrenic patients has also been found (Abi-Dargham, A. et al. 2000 PNAS USA 97:8104-8109) as has other evidence of presynaptic upregulation of DA metabolism (Meyer-Lindenberg, A. et al. 2002 Nat Neurosci 5:267-271). To the extent that schizophrenia involves both abnormalities in prefrontal dopamine signaling and in nigrostriatal dopamine activity, COMT genotype appears to contribute risk to each of these elements of the disorder and in the specific directions associated with the illness. The reciprocal effects of COMT genotype on DA signaling in the prefrontal cortex and on TH gene expression in the SN implicates a mechanism by which inheritance of COMT Val increases risk for schizophrenia and possibly other psychotic disorders.

Applicant assigns SEQ ID NO: 7 to the nucleic acid sequence for the human COMT gene and SEQ ID NO: 8 to the amino acid sequence for the human COMT protein set forth in Genbank Accession Number Z26491.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A method of detecting impaired prefrontal cognitive function in an individual comprising determining the individual's COMT genotype, and associating a high activity Val allele with impaired prefrontal cognitive function (and a low activity Met allele with enhanced prefrontal cognitive function).

2. A method of detecting impaired prefrontal cognitive function in an individual as indicative of a susceptibility to, or the presence of, a human condition that involves deficits in prefrontal cognitive function comprising determining the individual's COMT genotype, and associating a high activity Val allele with impaired prefrontal cognitive function as indicative of a susceptibility to, or the presence of, said human condition (and a low activity Met allele with enhanced prefrontal cognitive function as not indicative of a susceptibility to, or the presence of, said human condition).

3. A method of detecting impaired prefrontal cognitive function in an individual as predictive of improved prefrontal cognitive function upon administration of a COMT inhibitor or its pharmaceutically acceptable salt or ester comprising determining the individual's COMT genotype, and associating a high activity Val allele with impaired prefrontal cognitive function as predictive of improved prefrontal cognitive function upon administration of a COMT inhibitor or its pharmaceutically acceptable salt or ester (and a low activity Met allele with enhanced prefrontal cognitive function as not predictive of improved prefrontal cognitive function upon administration of a COMT inhibitor or its pharmaceutically acceptable salt or ester).

4. The method of any of claims 1-3, wherein said human condition is a member selected from the group consisting of Parkinson's Disease, AIDS, normal aging, brain injury, alcoholism, schizophrenia, depression, obsessive-compulsive disorder, attention deficit hyperactivity disorder, autism, impulse control disorder, addiction, Alzheimer's disease and other forms of dementia, mental retardation, and normal cognition.

5. The method of any of claims 1-3, wherein the determination of said individual's COMT genotype comprises detecting the presence of a COMT allele in an assay using a probe or primer comprised of an oligonucleotide that hybridizes to a sense or antisense sequence of the COMT gene set forth in GenBank Accession Number Z26491, or allelic variant, or 5′ or 3′ flanking sequences naturally associated with said COMT gene.

6. The method of claim 5, wherein said probe or primer is a probe attached to a DNA probe array.

7. The method of any of claims 1-3, wherein the determination of said individual's COMT genotype comprises detecting the presence of a COMT protein in an immunoassay using an antibody that is specifically immunoreactive with an allelic variant.

8. The method of any of claims 1-3, wherein the determination of said individual's COMT genotype comprises measuring performance in a neuropsychological test of executive cognition that is related to function of prefrontal cortex.

9. The method of claim 8, wherein said neuropsychological test is the Wisconsin Card Sorting Test or the N-back Task.

10. The method of any of claims 1-3, wherein the determination of said individual's COMT genotype comprises providing a probe or primer comprised of an oligonucleotide that hybridizes to a sense or antisense sequence of the COMT gene set forth in GenBank Accession Number Z26491, or allelic variant, or 5′ or 3′ flanking sequences naturally associated with said COMT gene, contacting the probe or primer with an appropriate nucleic acid containing sample, and detecting, by hybridization of the probe or primer to the nucleic acid, the presence or absence of a COMT allele.

11. The method of claim 10, wherein said nucleic acid containing sample is a blood sample.

12. The method of any of claims 1-3, further comprising administering to said individual a COMT inhibitor or its pharmaceutically acceptable salt or ester.

13-30. (canceled)
